(19) United States
(12) Patent Application Publication
(10) Pub. No.: US 2026/0062744 A1
(43) Pub. Date: Mar. 5, 2026

(54) COMPOSITIONS, SYSTEMS, AND METHODS FOR DETECTION OF METALS

(71) Applicant: MIAMI UNIVERSITY, OXFORD, OH (US)

(72) Inventors: KEVIN YEHL, DRY RIDGE, KY (US); KEVIN MORRISON, OXFORD, OH (US); MADELEINE TINCHER, OXFORD, OH (US)

(21) Appl. No.: 19/187,206

(22) Filed: Apr. 23, 2025

Related U.S. Application Data
(60) Provisional application No. 63/637,702, filed on Apr. 23, 2024.

(51) Int. Cl. C12Q 1/6874; G01N 33/487

(52) U.S. Cl. CPC C12Q 1/6874; G01N 33/48721

(57) ABSTRACT

Disclosed herein are compositions, systems, and methods for measuring analytes in a complex sample. Nanopore DNA zyme-based detection of certain metal contaminants is disclosed herein. Applicants constructed a multi-DNAzyme array and used a pattern-based readout to improve sensor accuracy. Applicants measured cross-reactivity between a variety of metal cofactors and common interferents and then used kinetic data to quantify the competing metal ion present in solution. The general inventive concepts provide means for detecting complex mixtures of analytes/metals in samples without the need of complex instrumentation.

### CROSS-REFERENCE TO RELATED APPLICATIONS

This application claims priority to and the benefit of U.S. Provisional Application No. 63/637,702, filed on Apr. 23, 2024, the content of which is incorporated by reference herein.

### FIELD

The present disclosure relates to compositions for and methods of monitoring levels of certain analytes of interest, including metal ions.

### SEQUENCE LISTING

The instant application contains a Sequence Listing which has been filed electronically in XML format and is hereby incorporated by reference in its entirety. Said XML copy, created on Aug. 29, 2025, is named 27876.04090 and is 56,398 bytes in size.

### BACKGROUND

Heavy metals, which include, lead, cadmium, mercury, and zinc, are pervasive environmental pollutants with detrimental effects on ecosystems and overall human health. Their abundant presence, coupled with bioaccumulation in the food chain and toxic nature to humans at minute concentrations, underscores the need for precise and reliable analytical methods to assess and monitor contamination levels to aid in preventing human exposure. The importance of accurate heavy metal detection cannot be overstated.

Analytical methods play a pivotal role in correctly identifying and quantifying heavy metals in various matrices, including water, soil, and biological samples. Flame Atomic Absorption Spectrometry (Flame AA), Inductively Coupled Plasma Spectroscopy equipped with either Atomic Emission Spectrometry (ICP-AES) or Mass Spectrometry (ICP-MS), have emerged as foundational techniques due to their sensitivity and versatility in detection. However, the selection of an appropriate method depends on factors such as the type of sample, desired detection limits, and cost considerations.

The simplicity, speed, sensitivity, and cost-effectiveness of Flame AA makes it an attractive choice for routine analysis in environmental, clinical, and industrial laboratories. Though Flame AA does have its advantages, it is not without limitations. One significant challenge is the potential for interference from matrix components. As a result, in many cases Flame AA can only look at one metal at a time depending on the system components. Another limitation of Flame AA is the technique's lower sensitivity compared to more advanced methods.

ICP-AES is another methodology for determining metal concentrations but it too is not immune to limitations. Although there is improvement in spectral line separation relative to Flame AA, it requires substantially more energy and is still susceptible to spectral interferences due to matrix effects, which can impact the accuracy of analytical results. To overcome these drawbacks with ICP-AES, mass spectrometry (MS), has recently been coupled with ICP to help decrease spectral interference due to its strong ability to determine oxidation states

ICP-MS provides several benefits for metal detection with significantly lower limit of detection (LOD) compared to both Flame AA and ICP-AES, but it is likewise not exempt from limitations. Many of these limitations parallel those of Flame AA and ICP-AES, including the need for a centralized laboratory, instrument costs, and the lack of portability. In addition, ICP also suffers from long start up times, and due to the amount of data MS can provide, it suffers from long analysis times. The general inventive concepts seek to overcome these and other drawbacks associated with the detection of metal ions.

### SUMMARY

The general inventive concepts are based, in part, on the discovery that DNAzyme-based biosensors offer several advantages over traditional methods such as Flame-AA, ICP-AES, and ICP-MS. Their two primary advantages are specificity and portability. As DNAzymes are discovered through an in vitro selection process akin to SELEX, they can be tailored to exhibit higher or lower specificity for a given metal, compared to the nearly fixed specificity of ICP-MS. This flexibility in specificity makes them ideal for use in a pattern-based array format for detection of metals in complex matrices (e.g., multiple metals) in a relatively small number of reactions that can be done on a plate. In such a system, especially with binary sensors, the array's capacity to detect unique patterns, corresponding to different metals, equals 2, where N is the number of sensor units in the array.

Traditional DNAzyme sensors function where substrate hydrolysis results in a signal response. For example, for a fluorescence readout, the reporter substrate is functionalized with a fluorophore and quencher, where hydrolysis results in the fluorophore being separated from the quencher, and thus an increase in fluorescence. Other conventional readouts include colorimetric and electrochemical.

The general inventive concepts are also based, in part, on the recognition that parallel detection of analytes (e.g., metals) in a sample is bottled-necked by detection technology. Current detection and measurement methodologies often rely on fluorescence-based detection. While relatively inexpensive, widely available, and well-understood, these and other common detection means suffer from critical drawbacks. In the case of fluorescence detection, what is required is different (often orthogonal) fluorophores to avoid spectral cross-over and achieve proper sensitivities for multiple analytes. Thus, detection is limited by available fluorophores and their spectral distance from one-another. The general inventive concepts are based on the discovery that DNA “barcoding” allows for accurate determination of analyte concentrations without the drawbacks associated with e.g., fluorescence-based detection. This coupled with DNAzyme technology and means for rapid sequencing provides compositions, systems, and methods for field-deployable, highly accurate, parallel detection of multiple analytes from a single sample.

Disclosed herein are compositions, systems, and methods for detecting the presence and/or concentration of analytes of interest (e.g., metals such as those of biological, environmental, or other significance) including DNAzyme-based detection and measurement.

In certain embodiments, the general inventive concepts are directed to a field depolyable system for real-time detection of a plurality of metal ions in a sample, the system comprising a DNAzyme array comprising a plurality of DNAzyme sequences sensitive to at least one predetermined metal ion, wherein each DNAzyme sensor comprises a DNAzyme region, a barcoded region, an optional detection enhancement sequence (in certain embodiments, it is a 200 bp segment added for detection), and a removal tag, means for removing unreacted DNAzyme substrate, and a nanopore sequencing device. In certain exemplary embodiments, the DNAzyme sequences sensitive to at least one predetermined metal ion comprises a RNA cleaving DNAzyme hybridized to a reporter containing a ribonucleotide across from a catalytic core of the DNAzyme. In certain exemplary embodiments, the barcoded region comprises a unique nucleotide sequence.

In certain embodiments, the general inventive concepts are directed to a method of detecting at least one metal ion in a sample. The method comprises obtaining a sample, contacting the sample with a DNAzyme array to form a metal assay mixture, optionally adding a means for removing unreacted DNAzyme substrate, sequencing the metal assay mixture to generate sample sequence data, comparing the sample sequence data to one or more predetermined metal profiles, and determining the presence of one or more metal ions in the sample based on the predetermined metal profiles.

Other aspects and features of the general inventive concepts will become more readily apparent to those of ordinary skill in the art upon review of the following description of various exemplary embodiments in conjunction with the accompanying figures.

### BRIEF DESCRIPTION OF THE DRAWINGS

The general inventive concepts, as well as embodiments and advantages thereof, are described below in greater detail, by way of example, with reference to the drawings in which:

FIG. 1 is an image of a general reaction scheme of an arbitrary DNAzyme that is sensitive to a metal cofactor, Mⁿ⁺, with a fluorescently reporting substrate strand.

FIG. 2 is a schematic of an embodiment of sequencing as a signal (here, production and analysis of an exemplary nanopore sequence).

FIG. 3A is a schematic showing heavy metal ion detection using DNAzyme sensors, where the metal ion of interest (Mn+) binds to the catalytic core (colored blue) of the DNAzyme, resulting in hydrolysis of the ribonucleotide linkage of the fluorescently quenched reporter substrate strand, resulting in an increase of fluorescence.

FIG. 3B is a schematic showing a DNAzyme array consisting of N DNAzymes interacting with N metals and demonstrative kinetic data.

FIG. 3C shows demonstrative response data for a DNAzyme array comprised of three DNAzymes.

FIG. 3D is a theoretical plot showing the scalability of a DNAzyme array compared to the traditional one DNAzyme sensor test approach, assuming a binary response.

FIG. 4 is a graphical illustration of an exemplary DNAzyme array constructed and tested as described herein. In this embodiment, five DNAzymes are employed to measure the cross-reactivity of eight metals at varying concentrations. The figure also shows a schematic showing the experimental approach and resulting output data.

FIG. 5A shows five exemplary DNAzymes used in an array with their associated metal cofactor.

FIG. 5B is a schematic comparing the original DNAzyme-substrate architectures to the universal substrate design according to the general inventive concepts. The DNAzyme catalytic core is omitted for simplicity and is represented by dashes. Black corresponds to the DNAzyme, green corresponds to the substrate, red corresponds to the ribonucleotide, purple corresponds to the universal substrate.

FIG. 5C is a kinetic plot for the 17E DNAzyme with (light green) and without (dark green) the metal cofactor (i.e., a predetermined metal ion) under each DNAzyme's reported buffer condition.

FIG. 5D a kinetic plot for the GR-5 DNAzyme with (light green) and without (dark green) the metal cofactor under each DNAzyme's reported buffer condition.

FIG. 5E is a kinetic plot for the EtNa DNAzyme with (light green) and without (dark green) the metal cofactor under each DNAzyme's reported buffer condition.

FIG. 5F is a kinetic plot for the Ag10c DNAzyme with (light green) and without (dark green) the metal cofactor under each DNAzyme's reported buffer condition.

FIG. 5G is a kinetic plot for the Na43 DNAzyme with (light green) and without (dark green) the metal cofactor under each DNAzyme's reported buffer condition.

FIG. 6A is heat map of endpoint fluorescence ΔF/F₀ at eight hours. Hashed out regions denote a combination where the competing metal and native metal are the same and were not measured. The * denote a precipitate was observed. The heat map was conditionally formatted per DNAzyme, where red denotes the lowest response, yellow is the 50th-percentile response, and green is the highest response observed for a particular DNAzyme.

FIG. 6B is a heat map of initial reaction velocity (t=0-15 mins) of the calibration data set.

FIG. 6C shows t-SNE dimension reduction of the calibration set where the competing metal and concentration were labeled. The concentration of native metal, endpoint ΔF/F₀ and initial reaction velocity are the signal features.

FIG. 6D shows k-Means clustering analysis of the t-SNE dimension reduced data.

FIG. 7A is a plot of t-SNE analysis for the 200 mM Pb² validation solution. Insets highlight the region where the validation solutions cluster.

FIG. 7B is a plot of t-SNE analysis for the 2 mM Pb²⁺, validation solution. Insets highlight the region where the validation solutions cluster.

FIG. 7C is a plot of t-SNE analysis for the 200 μM Ag⁺, validation solution. Insets highlight the region where the validation solutions cluster.

FIG. 7D is a plot of t-SNE analysis for the 200 100 μM Zn²⁺ validation solution. Insets highlight the region where the validation solutions cluster.

FIG. 7E summary of the top 20 hits based on RMSE of the initial reaction velocity for the validation solution compared to the calibration set for 2 mM Pb²⁺.

FIG. 7F is a summary of the top 20 hits based on RMSE of the initial reaction velocity for the validation solution compared to the calibration set for 200 mM Pb²⁺.

FIG. 7G is a summary of the top 20 hits based on RMSE of the initial reaction velocity for the validation solution compared to the calibration set for 200 μM Ag⁺.

FIG. 7H is a summary of the top 20 hits based on RMSE of the initial reaction velocity for the validation solution compared to the calibration set for 100 μM Zn²⁺.

FIG. 8 is a graphical abstract showing the conversion of the fluorescent reporter architecture to the DzNanoporeSeq architecture.

FIG. 9(3.2) is a schematic outlining the construction of the DzNanoporeSeq reporter architecture and experiment.

FIG. 10A shows a sequence schematic comparing the original DNAzyme-substrate architectures to the universal substrate design, where the catalytic core was replaced with - - - -.

FIG. 10B shows a sequence schematic showing the construction of the DzNanoporeSeq substrate, where most of the Broc DNA was replaced with - - - -.

FIG. 11A is a schematic outlining the biotin binding experiment.

FIG. 11B shows the Biotin-Streptavidin binding fluorescent kinetics for the biotinylated universal fluorescent substrate to streptavidin.

FIG. 12A is a graph showing a time study of nanopore sequencing for 185.5 fmol (dynamic range: 21, 25, 29, 33, 37.5, and 40 fmol) formulation.

FIG. 12B is a graph showing a time study of nanopore sequencing for 57.9 fmol (dynamic range: 0.15, 1.5, 3.75, 7.5, 15, and 30 fmol) formulation.

FIG. 12C is a graph showing a time study of nanopore sequencing for 38.875 fmol (dynamic range: 0.125, 1.25, 2.5, 5, 10, and 20 fmol) formulation.

FIG. 12D is a graph showing a time study of nanopore sequencing for 19.3 fmol (dynamic range: 0.05, 0.5, 1.25, 2.5, 5, and 10 fmol) formulation.

FIG. 12E is a graph showing a time study of nanopore sequencing for

FIG. 12F is a graph showing the calibration curve of raw counts of DNA for a particular barcode at 12 hrs.

FIG. 13 shows, from left to right, graphs of the calibration curves ascertained by DzNanoporeSeq, fluorescence spectroscopy, and comparison of DzNanoporeSeq to fluorescence spectroscopy. Error bars represent SEM (DzNanoporeSeq: n=2, Fluorescent Spectroscopy: n=3). The line-of-best fit for Lit by DzNanoporeSeq: y=0.3733+0.3229 (R²=0.9999); F/F₀: y=0.0291x+0.9914 (R²=0.9999); DzNanoporeSeq compared to F/F₀: y=10.791x−8.2445 (R²=0.9997). The line-of-best fit for Ca²⁺ by DzNanoporeSeq: y=0.3601+0.3436 (R²=0.9999); F/F₀: y=0.0146x+1.0321 (R²=0.9952); DzNanoporeSeq compared to F/F₀: y=23.24x−22.181 (R²=0.9967). The line-of-best fit for Pb²⁺ by DzNanoporeSeq: y=59.762-57.183 (R²=0.9978); F/F₀: y=0.0070x+1.0071 (R²=0.9993); DzNanoporeSeq compared to F/F₀: y=0.6480x−0.6468 (R²=0.9999).

FIG. 14 is a flowchart showing an exemplary method of determining the presence of a predetermined metal ion in a sample.

FIG. 15 shows an exemplary computing device for use according to the general inventive concepts.

FIG. 16 is a table with a summary of all DNA sequences used in DzNanoporeSeq studies.

### DETAILED DESCRIPTION

Several illustrative embodiments will be described in detail with the understanding that the present disclosure merely exemplifies the general inventive concepts. Embodiments encompassing the general inventive concepts may take various forms and the general inventive concepts are not intended to be limited to the specific embodiments described herein.

While various exemplary embodiments are described or suggested herein, other exemplary embodiments utilizing a variety of methods and materials similar or equivalent to those described or suggested herein are encompassed by the general inventive concepts.

Unless defined otherwise, all technical and scientific terms used herein have the same meaning as commonly understood by one of ordinary skill in the art to which the invention belongs.

The terms “DNAzyme-metal sample” and “metal assay mixture” refer to the combination of a DNAzyme array according to the general inventive concepts and a sample (e.g., a water sample form an area of interest). The mixture generally includes all of the necessary components for reaction between a DNAzyme and a metal ion as described herein. In certain embodiments, the sample or mixture may also include a means for removing unreacted substrate (e.g., streptavidin coated beads when the DNAzyme sensor comprises biotin, as described herein. Those of ordinary skill in the art will recognize that while the pairing of streptavidin magnetic beads and biotin is disclosed herein, other systems for coordination and sequestration of unreacted DNAzyme can also be used and would still fall under the general inventive concepts) and other additives or adjuvants to improve sensitivity or detection of the analyte of interest (e.g., a heavy metal ion).

The term “DNAzyme array” refers to a collection or plurality of DNAzymes that are collectively sensitive to a plurality of metal ions and that are used to generate a metal ion profile, according to the general inventive concepts.

The term “predetermined metal ion” refers to a metal co-factor that catalyzes a DNAzyme to cleave an RNA substrate according to the general inventive concepts. Those of ordinary skill will recognize that ligation DNAzymes also fall within the general inventive concepts. In certain exemplary embodiments, the predetermined metal ion is a metal ion of particular interest for environmental, biological, or human health-related reasons. In certain exemplary embodiments, the predetermined metal ion is selected from the group consisting of Li⁺, Na⁺, Ag⁺, UO₂²⁺, Cu²⁺, Cd²⁺, Ca²⁺, Hg²⁺, Mn²⁺, Pb²⁺, Tl³⁺, Ln³⁺, Cr³⁺, Cr⁶⁺, Ni²⁺, As³⁺, La³⁺, Ce³⁺. Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, and Lu³⁺.

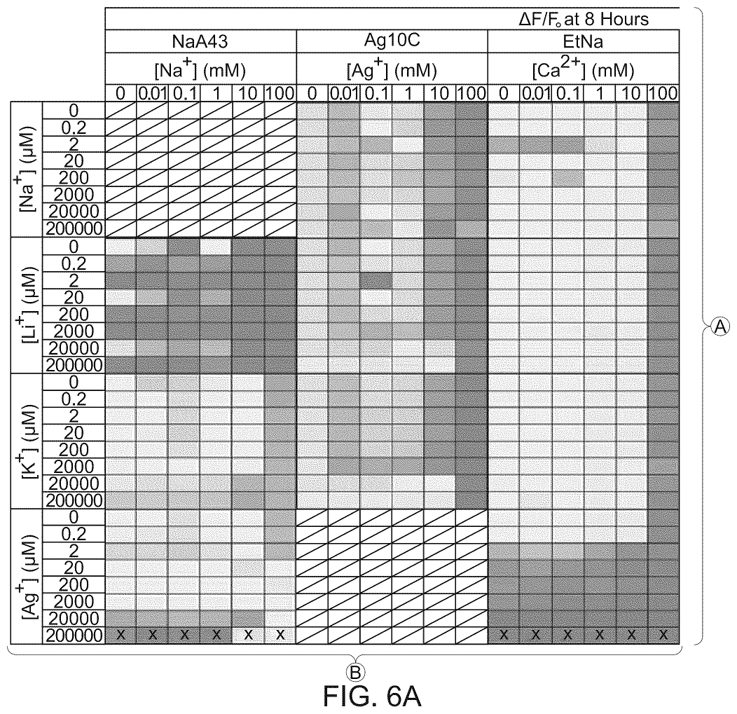

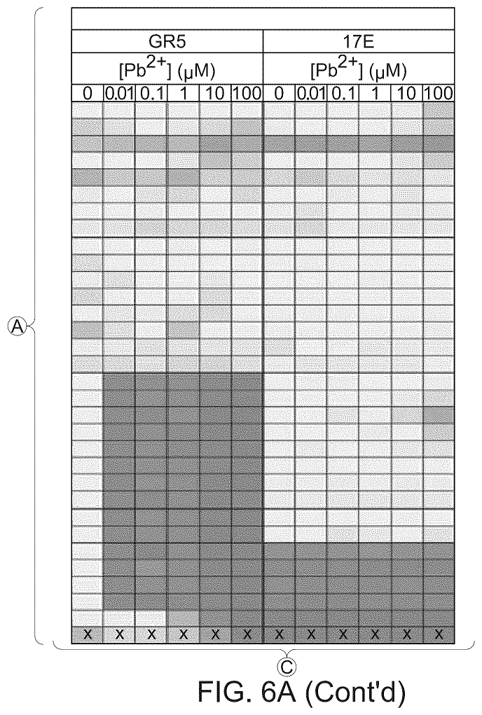

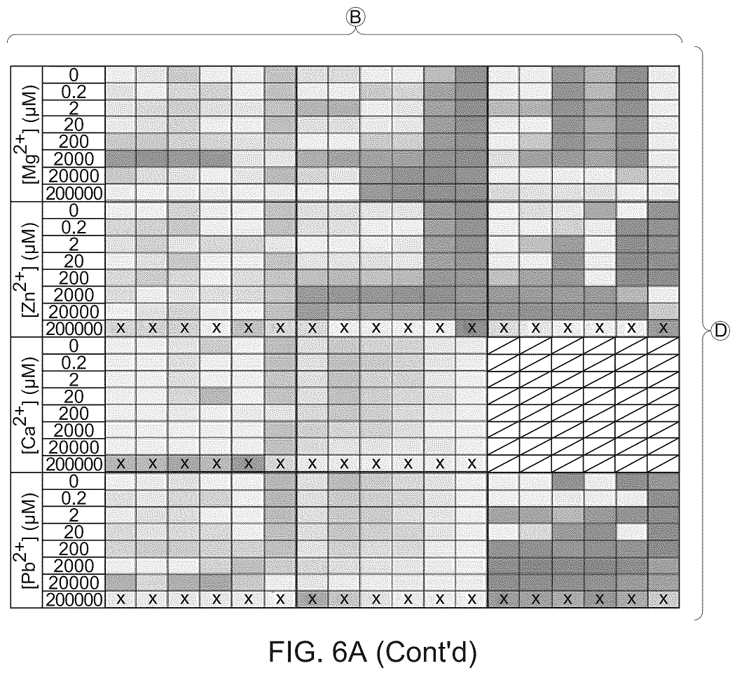

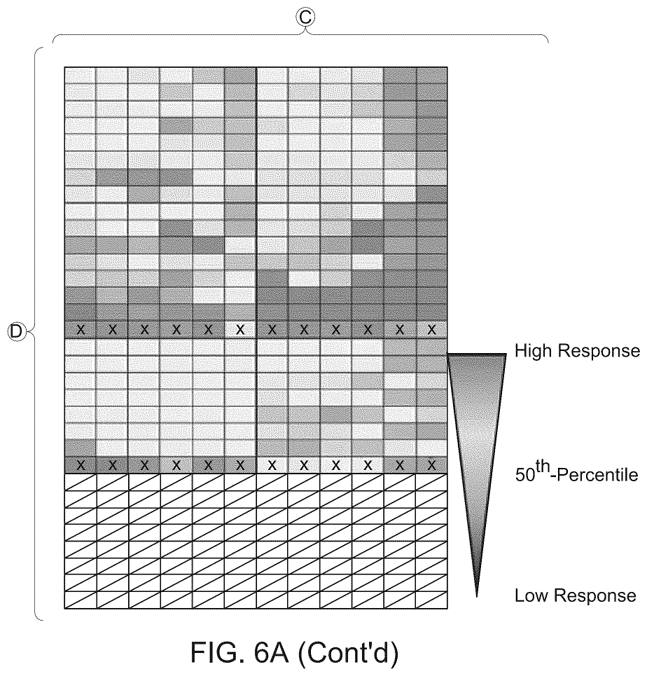

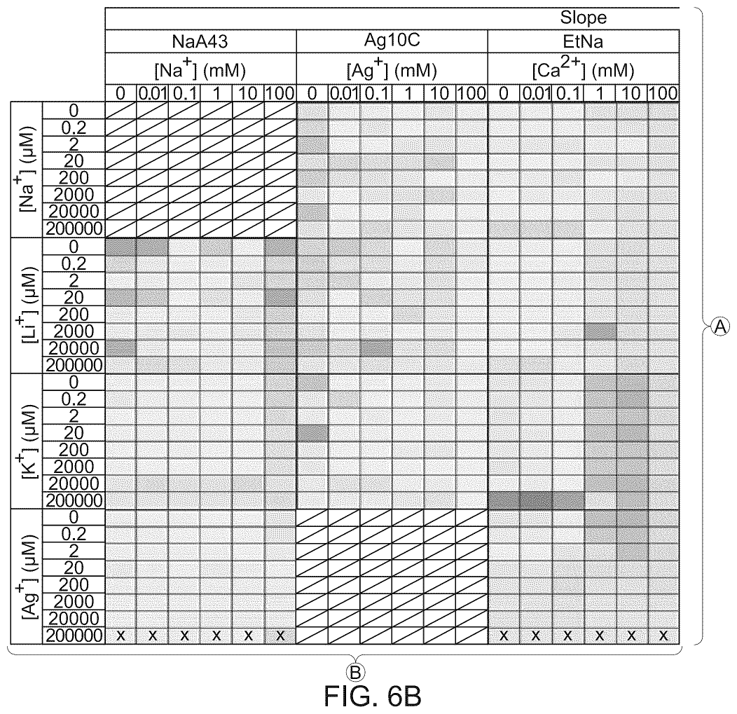

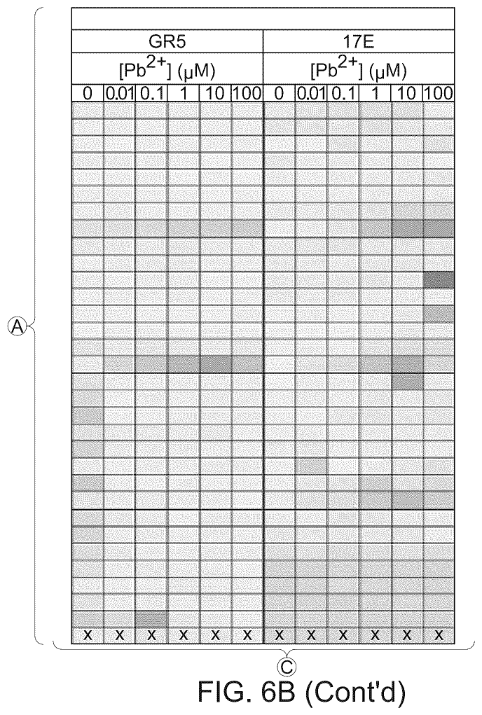

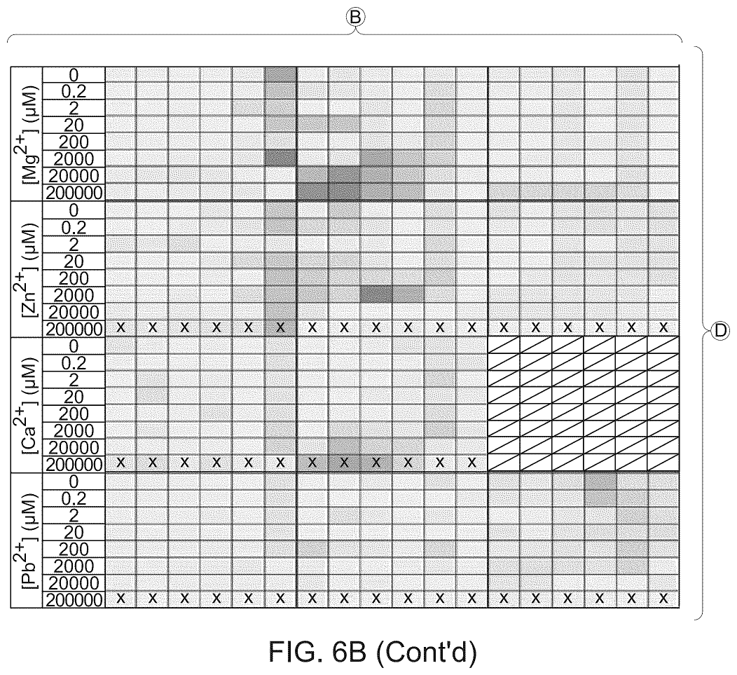

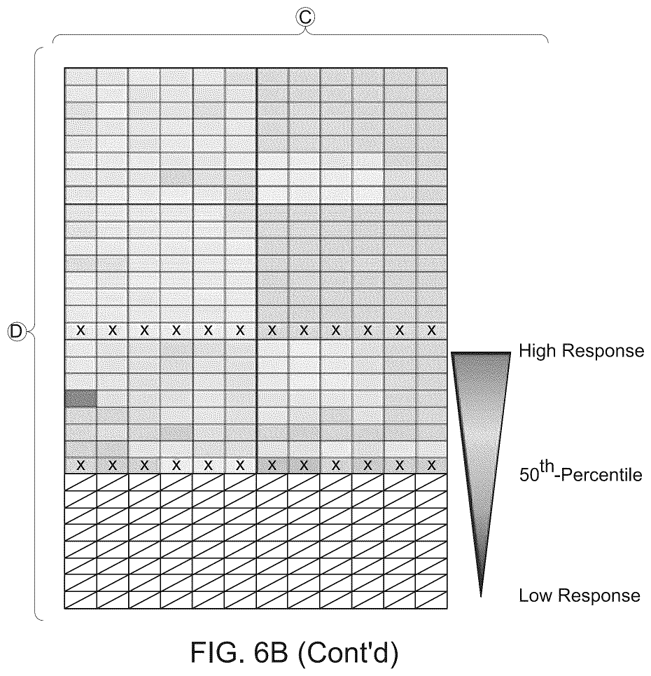

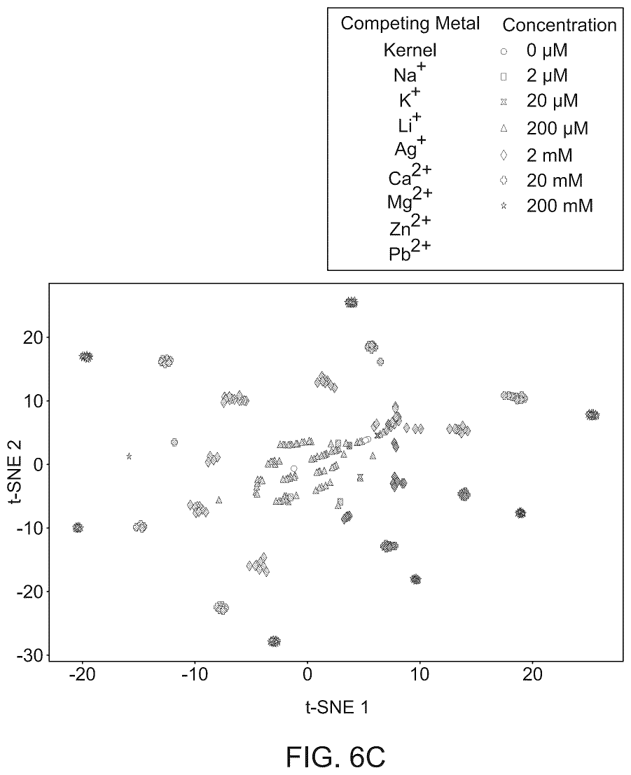

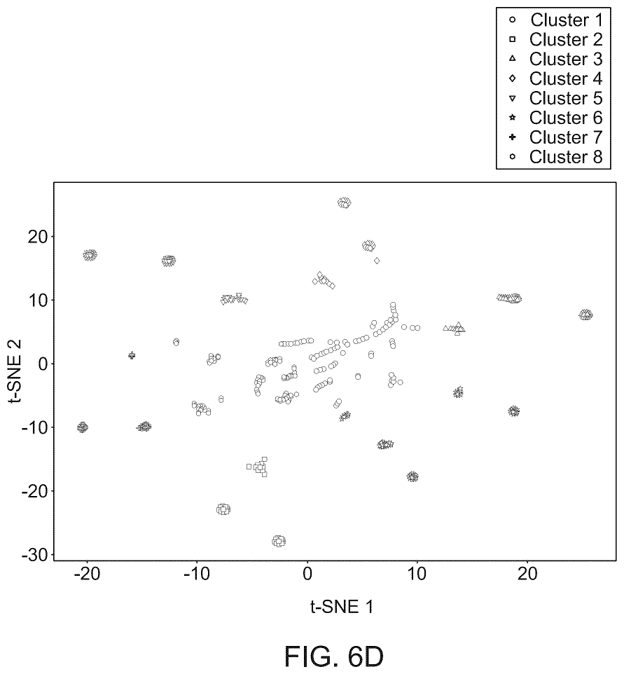

The term “metal ion profile” refers to a unique fingerprint or pattern of relative reactivity of one or more metal ions when reacted with an array/plurality of DNAzyme sensors. The individual reactivity of a metal ion toward the particular DNAzyme sensors creates a unique pattern of activity for each metal (See e.g., FIG. 6 and accompanying discussion). In certain exemplary embodiments, the metal ion profile is a grid based on heat mapping of at least two DNAzyme sensors' kinetic data. In certain embodiments, the metal ion profile or measurement is determined by an array by sequencing a sample, and determining a readout by carrying out multiple reactions (barcoded) and then readout their ratios.

The term “sensitive to” when referring to DNAzyme sensors refers to the predetermined metal ion for which the DNAzyme was predominantly designed or modified to react with. In other words, a DNAzyme is sensitive to a predetermined metal ion if it catalyzes a reaction as described herein to produce a signal sequence according to the general inventive concepts. In certain embodiments, a DNAzyme sensor need not necessarily be sensitive to only one predetermined metal ion, and in certain embodiments, a particular DNAzyme is sensitive to only one predetermined metal ion.

The term “removal tag” as used herein refers to a portion of the DNAzyme sensor that has a moiety incorporated for the purpose of removing unreacted DNAzyme, to e.g., increase detection sensitivity or reduce noise. The tag may also be referred to as a pull down sequence. In certain exemplary embodiments, the removal tag may be a biotin-containing sequence that is paired with e.g., streptavidin beads for removing unreacted DNAzyme sensor from a sample (i.e., an embodiment of a means for removing unreacted DNAzyme substrate), prior to sequencing.

Applicants herein demonstrate the design, fabrication and characterization of systems for determining the presence and/or concentration of at least one analyte of interest, including predetermined metal ion(s).

The general inventive concepts are based, in part, on the discovery that DNAzyme-based biosensors offer several advantages over traditional methods of metal detection in a sample. (See also Morrison, K. S.: Innovative approaches for multiplext detection: DNAzyme and bacteria-based arrays for environmental monitoring, Doctoral dissertation, Miami University (2024)) the content of which is hereby incorporated herein by reference in its entirety). Conventional DNAzyme sensors function where substrate hydrolysis results in a signal response. For example, for a fluorescence readout, the reporter substrate is functionalized with a fluorophore and quencher, where hydrolysis results in the fluorophore being separated from the quencher, and thus an increase in fluorescence.

The general inventive concepts are also based, in part, on the recognition that parallel detection of analytes (e.g., metals) in a sample is bottled-necked by detection technology. Current detection and measurement methodologies often rely on fluorescence-based detection. But detection is then limited by available fluorophores and their spectral distance from one-another. The general inventive concepts are based on the discovery that DNA “barcoding” allows for accurate determination of analyte concentrations without the drawbacks associated with e.g., fluorescence-based detection. This coupled with DNAzyme technology and means for rapid sequencing provides compositions, systems, and methods for field-deployable, highly accurate, parallel detection of multiple analytes (i.e., metal ions) from a single sample. Further, the general inventive concepts are not necessarily limited by the type of sample. In certain exemplary embodiments, the sample is an aqueous or otherwise liquid sample (e.g., from a water source or treatment plant). In certain embodiments, the sample is a solid sample (e.g., a soil sample) that may be diluted with an appropriate solvent or water, filtered to remove unwanted solids, and then diluted with the appropriate solvent or buffer for sequencing.

While Applicants have demonstrated the effectiveness of a particular set of DNAzymes in an array for the detection of at least one predetermined metal ion, those of ordinary skill in the art will recognize that the general inventive concepts may be extended to metal ions and DNAzymes other than those expressly disclosed herein without departing from the scope and spirit of the general inventive concepts.

Heavy metal contamination of food and water poses a significant public health risk due to the potential for acute toxicity at minute concentrations. DNAzyme sensors offer a promising solution for rapid on-site heavy metal detection to limit exposure, but their widespread deployment is hindered by limitations in multiplexing capabilities, especially when using fluorescent sensor constructs. To address this challenge, Applicants assembled a novel nanopore sequencing reporter substrate for parallel analysis of three DNAzymes (GR-5, EtNa, and 20-4) in the presence of their respective metal co-factors (e.g., a predetermined metal ion such as Pb²⁺, Ca²⁺, and Li⁺). Comparative analysis with the conventional fluorescence method revealed a 4× higher specificity. These findings represent a significant advancement in DNAzyme sensors, offering a pathway towards the development of highly multiplexed arrays.

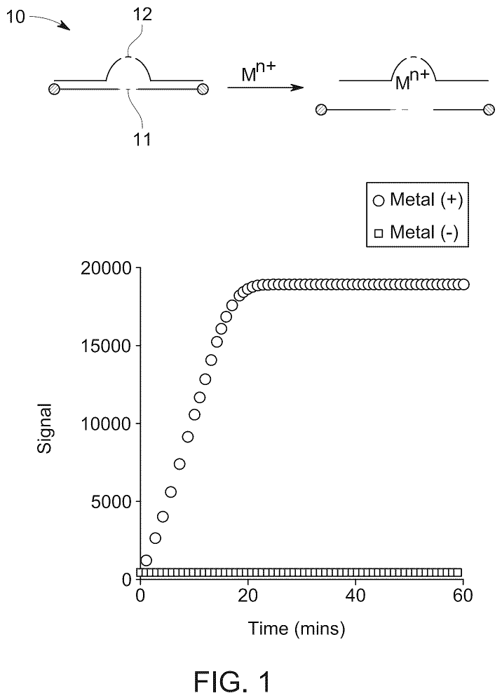

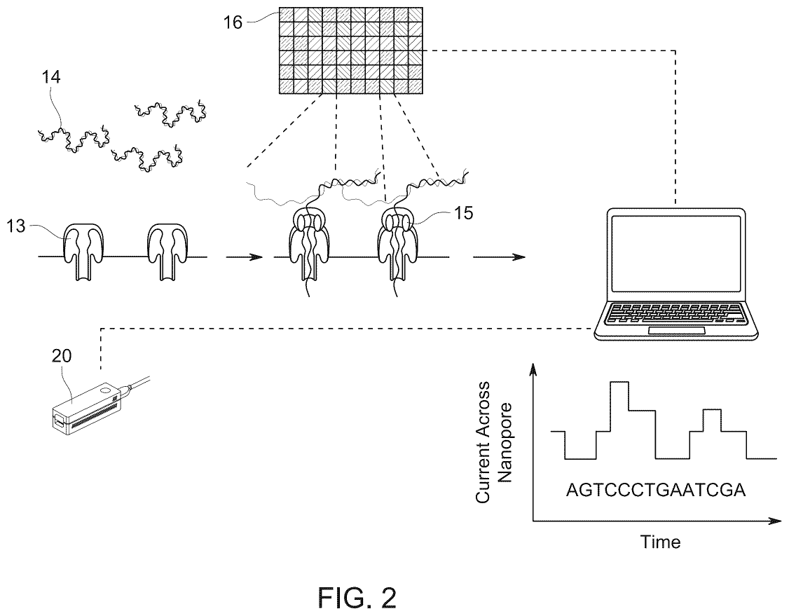

DNAzymes are a class of functional nucleic acids (FNA) that have catalytic activity and are also referred to as catalytic nucleic acids (CNAs). Originally discovered from basic science research as a ribozyme mimetic, this class of molecule has shown significant potential as heavy metal sensors, where the metal ion (Mⁿ⁺) is a cofactor for catalytic activity. The most common sensor architecture comprises an RNA cleaving DNAzyme (RCD) hybridized with a reporter substrate containing a ribonucleotide across from the catalytic core. In the presence of the respective metal cofactor, RNA hydrolysis occurs, resulting in an increase in signal, which can be colorimetric, fluorescent, luminescent, or electrical current depending on the reporter substrate design. FIG. 1 shows a general reaction scheme of a DNAzyme 10 that is sensitive to a predetermined metal ion (cofactor), M″, with a fluorescently reporting substrate strand. The ribonucleotide 11 and the catalytic core 12. A theoretical signal versus time plot for the arbitrary DNAzyme in both the presence and absence of metal is shown as well. In certain exemplary embodiments, the general inventive concepts contemplate systems and methods comprising a DNAzyme adapted for detecting a predetermined metal ion selected from Li⁺, Na⁺, Ag⁺, UO₂²⁺, Cu²⁺, Cd²⁺, Ca²⁺, Hg²⁺, Mn²⁺, Pb²⁺, Tl³⁺, Ln³⁺, Cr³⁺, Cr⁶⁺, Ni²⁺, As³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, and Lu³⁺, and in certain embodiments, the predetermined metal ion is selected from Li⁺, Na⁺, K⁺, Ag⁺, Mg²⁺, Ca²⁺, Zn²⁺, and Pb²⁺. In certain embodiments, the systems and methods of the general inventive concepts contemplate sensing any target molecule/analyte a DNAzyme can sense. For example, in certain embodiments, a DNAzyme may be adapted by combining DNAzymes and aptamers together to make an aptazyme. These sensors can detect small organic molecules and biomolecules that are not otherwise catalytically active. In certain embodiments, the general inventive concepts contemplate detection and determining the concentration of e.g., bacteria in a sample. FIG. 2 shows a schematic of an exemplary sequencing means. In a general sense, voltage is applied across a nanopore. The DNA is threaded through a nanopore which results in current blockage and an electrochemical readout for the sequence. In certain exemplary embodiments, (e.g., in FIG. 2), the sequencing means is a nanopore sequencer 20 (here, shown attached to a processing means such as a laptop computer). From left to right, the membrane of the nanopore 13 has voltage placed across it so that the DNA 14 in solution is brought into contact. Once in contact with the nanopore, a motor protein 15 feeds the DNA into the nanopore which leads to a change in current observed by that specific pore. The change in current is ultimately processed through a basecaller software and can be identified as a nucleotide (bottom right image).

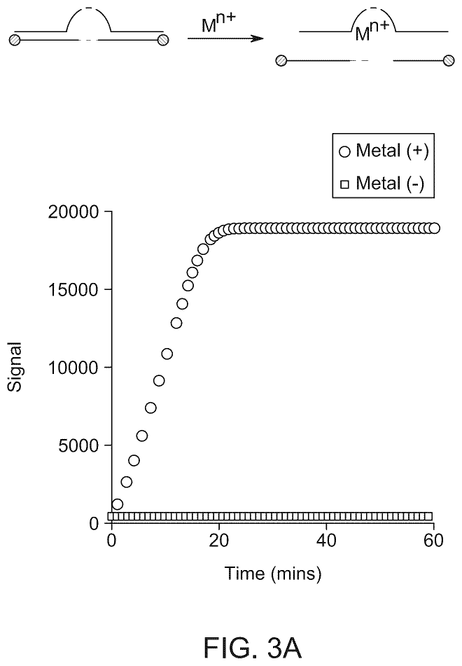

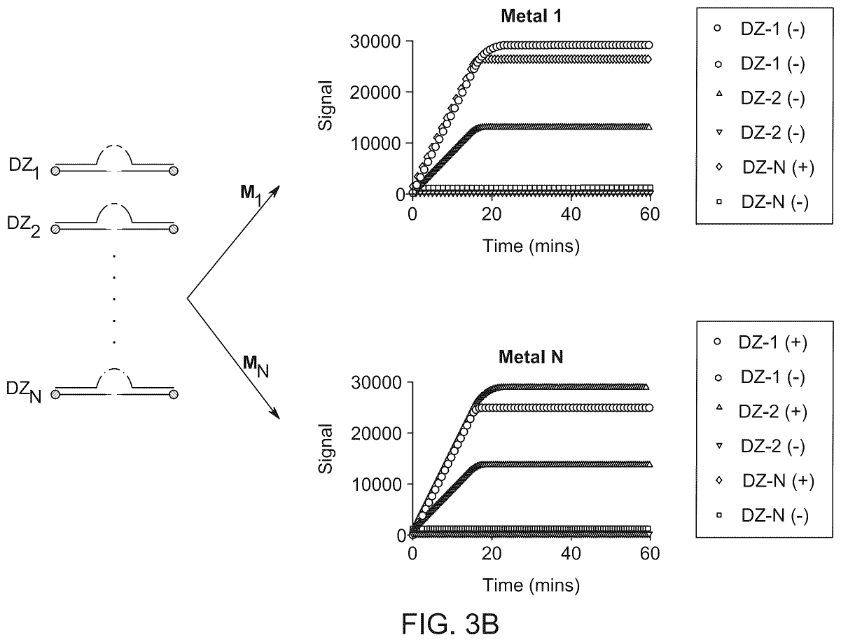

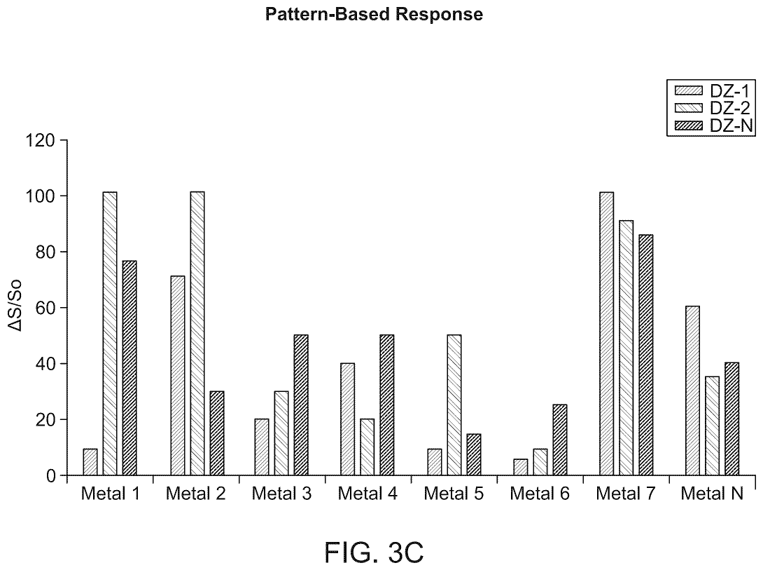

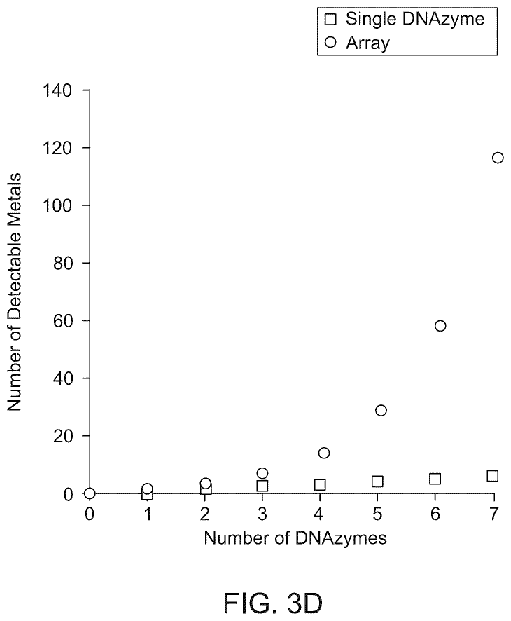

To date, several RCDs have been discovered to detect a multitude of metal ions including but not limited to: Li⁺, Na⁺, Ag⁺, UO₂²⁺, Cu²⁺, Cd²⁺, Ca²⁺, Hg²⁺, Mn²⁺, Pb²⁺, Tl³⁺, Ln³⁺, Cr³⁺, and Cr⁶⁺. From this list, targeted tests can be carried out using the respective DNAzyme sensor to detect the presence and/or concentration of one or more predetermined metal ions. However, this approach requires N arbitrary tests to detect N metal ions, thus scaling linearly, FIG. 3D. Furthermore, though most DNAzyme sensors are substantially more active in the presence of their respective metal cofactor, this selectivity is not absolute and often results in cross-reactivity with similar metals, FIGS. 3B and C. For example, DNAzyme 17E and GR-5 are two Pb²⁺-sensitive DNAzymes that have cross reactivities with Mg²⁺ and Zn²⁺. For 17E, 10 mM and 5 mM concentrations of Mg²⁺ and Zn²⁺, respectively, result in the same response as 10 μM and 200 μM Pb²⁺, respectively. Though GR-5 has much higher selectivity comparatively, 1 mM of Zn²⁺ still yields the same sensor response as 25 nM of Pb²⁺.

Applicants have surprisingly discovered that this heretofore undesirable cross-reactivity can be harnessed to improve sensor accuracy and even scale multiplex detection by using a pattern-based readout, 3C (i.e., to determine a metal ion profile or “fingerprint” for each predetermined metal ion). A pattern-based response enables scaling detection up to 2^{N} analytes that display unique patterns, where Nis the number of sensor array units where Nis the number of sensor array units assuming a binary sensor response, FIG. 3D. This approach can be viewed as an extension of the extensive body of literature showing that nonspecific sensor arrays can selectively detect a multitude of analytes with relatively few sensor elements.

Applicants confirmed the viability of this strategy using five DNAzymes (17E, GR-5, EtNA, Ag10c, and NaA43) and measuring their cross reactivity between their metal ion cofactor (Pb²⁺, Ca²⁺, Ag⁺, and Na⁺) and common interferents (Mg²⁺, Zn²⁺, Li⁺, and K²⁺). To generate a metal ion profile for select predetermined metal ions, even in the presence of common/known interferents.

One of the major limitations for the widescale deployment of DNAzymes for environmental monitoring is their limitation on multiplexing DNAzyme sensors together. For example, when using a fluorescent reporter substrate, the maximum number of DNAzymes that can be screened at one time is limited by the number of orthogonal fluorophores, i.e., fluorophores with no emission overlap. Since DNAzyme sensors are DNA-based, barcoding DNAzyme sensors and using a sequencing readout offers an interesting solution for the parallel analysis of multiple DNAzyme sensors in a single assay.

To barcode DNAzyme sensors, sensors are labeled with a unique nucleotide sequence, typically several base pairs long. This unique barcode is used to distinguish different DNAzyme sensors when they are sequenced together on the same flow cell. As a result, the maximum number of DNA samples that can be loaded onto a single flow cell and analyzed is limited by the number of orthogonal barcodes possible, 4^{length of barcode}, and by the read-count which is dependent on the sequencing platform. As a result, it is possible to screen several orders of magnitude more DNAzyme sensors in a single reaction compared to using a fluorescent signal. Lastly, with the advent of nanopore sequencing technologies, particularly the flongle flow cell technology from Oxford Nanopore (a flongle is an adapter for MinION or GridION that enables direct, real-time DNA sequencing, or cDNA sequencing on smaller, single-use flow cells), it is possible to perform sequencing without a centralized laboratory due to its portability and only needing a laptop to power and acquire data.

Herein, we report for the first time using a sequencing-based method (DzNanoporeSeq) for the detection of several metal ions (Li⁺, Ca²⁺, and Pb²⁺) using DNAzyme sensors (20-4: Li⁺-sensitive, EtNa: Ca²⁺-sensitive GR-5: Pb²⁺-sensitive) and multiplexing in a single test. We hypothesize that DzNanoporeSeq will unlock the multiplexing potential that DNAzymes offer.

In principle, the number of DNAzymes that can be multiplexed in a single assay is limited by the number of reads. Sequencing technologies can achieve up to one billion reads. This equates to ˜10 million possible DZ sensors that can be tested in parallel, assuming a hundred-fold coverage. This is an extremely large number of possibilities. Thus, multiplexing is now limited by the number of sensors.

Applicants constructed a multiplexed assay consisting of three DNAzymes for the detection of three metals of both environmental and biological significance. We compared this new method (referred to herein as DzNanoporeSeq) to the traditional fluorescent substrate reporter method and we were able to generate calibration curves from internal standards. By comparing the validation solution tests, we observed that DzNanoporeSeq yields more accurate concentrations for all validation solutions when compared to the conventional fluorescence spectroscopy methods, Table 2.

The results shown in Table 3 showed minimal variance between thresholds, which highlights the minimal variance and scalability of the methods disclosed herein. In addition, since the method used unique barcodes to tag each DNAzyme and metal concentration tested, assuming infinite number of reads, the number of DNAzymes being tested and the number of different concentrations, samples and thresholds being tested in a single assay are only limited by the number of unique barcodes, 4^{length of barcode}, which leads to an extremely large sampling landscape possible.

Although the DzNanoporeSeq highlighted numerous advantages, it is not without some disadvantages. One of these disadvantages was the sacrifice to sensitivity for the parallel analysis of DNAzyme sensors. In addition, another disadvantage is that in the current state of nanopore sequencing available from Oxford Nanopore Technologies, the motor protein, which threads the DNA into the nanopore, is attached to the adaptor sequence. As a result, the prepped DNA would have to be stored at −20° C. for long-term stability of sensor unit. Another disadvantage is that since the motor protein is on the adaptor, the adaptor sequence must be purchased from Oxford Nanopore, thus increasing the cost tremendously since the adaptor sequence cannot simply be synthesized for the sensor unit to be analyzed by nanopore sequencing. A possible solution around this is the transition from a biologic nanopore to a solid state nanopore, which do not utilize motor proteins to thread the DNA into the nanopore.

Applicants developed a universal substrate for DzNanoporeSeq to further reduce the assay costs that nanopore sequencing already provides. With convergence of sequencing technologies, increasing computational power, and advancements in machine learning algorithms, the field is now better positioned for the full realization of the power DNAzymes can provide for determination of the presence and/or concentration of one or more metal ions.

Further, Applicants also demonstrate that DNAzyme-based systems, due to their relative size and weight, can be field-deployed in complex environmental systems to detect and monitor the presence of analytes of interest (e.g., predetermined metal ions).

Accordingly, in certain exemplary embodiments, the general inventive concepts contemplate a DNAzyme array for use measurement of analytes of interest. Those of ordinary skill in the art will recognize that the general inventive concepts are not necessarily limited to DNAzymes, but may also employ aptamers and/or aptazymes to expand detection of multiple analyte classes in a single test, such as small molecules, lipids, nucleic acids, and proteins. In certain embodiments, the general inventive concepts are directed to a field depolyable system for real-time detection of a plurality of metal ions in a sample, the system comprising a DNAzyme array comprising a plurality of DNAzyme sequences sensitive to at least one predetermined metal ion, wherein each DNAzyme sequence comprises a DNAzyme region, a barcoded region, an optional detection enhancement sequence (the 200 bp segment added for detection), and a (biotin region), means for removing unreacted DNAzyme substrate, and a nanopore sequencing device.

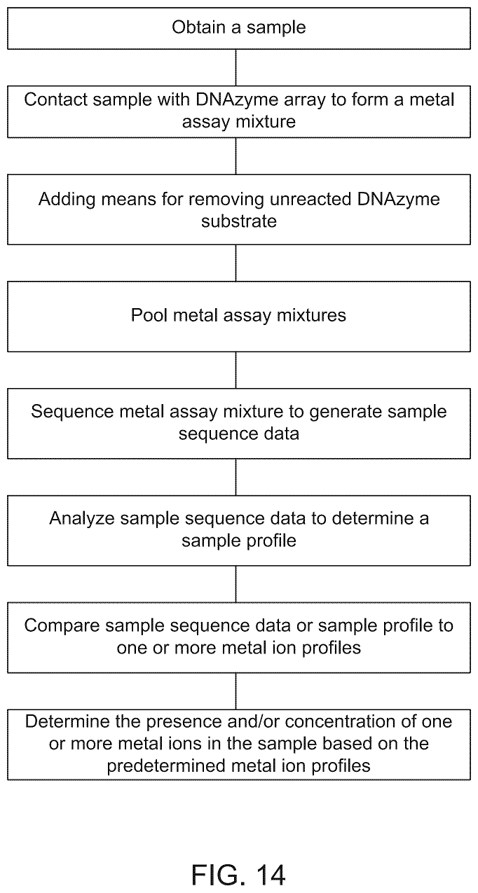

In certain embodiments, the general inventive concepts are directed to a method of detecting a plurality of metal ions in a sample. See FIG. 14, which is a flow chart showing an exemplary method of detecting at least one predetermined metal ion in a sample. The method comprises obtaining a sample, contacting the sample with a DNAzyme array to form a metal assay mixture, optionally adding a means for removing unreacted DNAzyme substrate, sequencing the metal assay mixture to generate sample sequence data, comparing the sample sequence data to one or more predetermined metal profiles, and determining the presence of one or more metal ions in the sample based on the predetermined metal profiles. In certain exemplary embodiments, contacting comprises allowing the sample and DNAzyme array to remain together in the mixture for a predetermined amount of time, including a sufficient amount of time for sufficient reaction between the metal ion of interest and the DNAzyme.

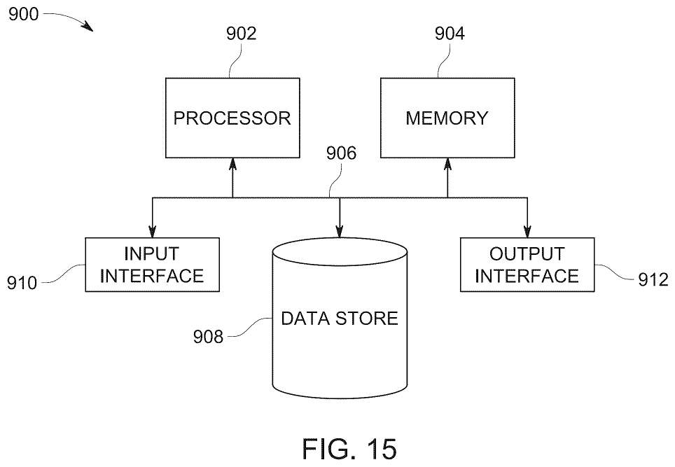

Referring now to FIG. 15, which shows a high-level illustration of an exemplary computing device 900 that can be used in accordance with the systems and methodologies disclosed herein is illustrated. For instance, the computing device 900 may be used in a system for measuring the presence of a metal ion in a sample. The computing device 900 includes at least one processor 902 that executes instructions that are stored in a memory 904. The instructions may be, for instance, instructions for implementing functionality described as being carried out by one or more components discussed above or instructions for implementing one or more of the methods described above. The processor 902 may access the memory 904 by way of a system bus 906.

The computing device 900 additionally includes a database 908 that is accessible by the processor 902 by way of the system bus 906. The data store 908 may include executable instructions and/or metal ion profile data.

The computing device 900 also includes an input interface 910 that allows external devices to communicate with the computing device 900. For instance, the input interface 910 may be used to receive instructions from an external computer device, from a user, etc. The computing device 900 also includes an output interface 912 that interfaces the computing device 900 with one or more external devices. For example, the computing device 900 may display text, images, etc. by way of the output interface 912.

It is contemplated that the external devices that communicate with the computing device 900 via the input interface 910 and the output interface 912 can be included in an environment that provides substantially any type of user interface with which a user can interact. Examples of user interface types include graphical user interfaces, natural user interfaces, AR interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 900 in a manner free from constraints imposed by input devices such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 900 may be a distributed system. Thus, for instance, several devices may be in communication by way of a network connection and may collectively perform tasks described as being performed by the computing device 900.

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. Disk and disc, as used herein, include compact disc (CD), laser disc, optical disc, digital versatile disc (DVD), floppy disk, and Blu-ray disc (BD), where disks usually reproduce data magnetically and discs usually reproduce data optically with lasers. Further, a propagated signal is not included within the scope of computer-readable storage media. Computer-readable media also includes communication media including any medium that facilitates transfer of a computer program or data from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

All references to singular characteristics or limitations of the present disclosure shall include the corresponding plural characteristic or limitation, and vice versa, unless otherwise specified or clearly implied to the contrary by the context in which the reference is made.

The following examples illustrate features and/or advantages of the compositions, systems, and methods according to the general inventive concepts. The examples are given solely for the purpose of illustration and are not to be construed as limitations of the general inventive concepts, as many variations thereof are possible without departing from the spirit and scope of the general inventive concepts.

### EXAMPLES

Materials and Reagents: DNAzymes and substrate oligonucleotides were purchased from IDT (Newark, USA). Lead (II) nitrate (ACS certified) and HEPES (BioPerformance Certified, ≥99.5%) were purchased from Sigma-Aldrich. Calcium nitrate tetrahydrate (ACS Certified), magnesium nitrate hexahydrate (ACS Certified), zinc nitrate hexahydrate (ACS Certified), silver nitrate (ACS Certified), and potassium nitrate (ACS Certified) were purchased from Fisher Chemicals. Sodium nitrate (ACS certified) was purchased from Alfa Aesar. Sodium hydroxide was purchased from Fisher BioReagents. Black round bottom 96-well plates were purchased from Fisher Scientific (Product #: 15-100-175).

### DNAzyme Cross Reactivity Studies

DNAzyme catalytic activity was determined by measuring the rate of hydrolysis of a DNA/RNA chimera substrate that was functionalized with a 5′ 6-fluorescein (FAM6) and a 3′ black hole quencher (BHQ). Hydrolysis was measured by monitoring fluorescence intensity as a function of time using a BioTek Synergy LX plate reader equipped with a tungsten lamp. Fluorescence measurements were taken every minute for eight hours at room temperature (24° C.) using a green filter (Ex. 485/20, Em. 528/20) and a gain of 50. In between measurements, plates were shaken with double orbital rotation.

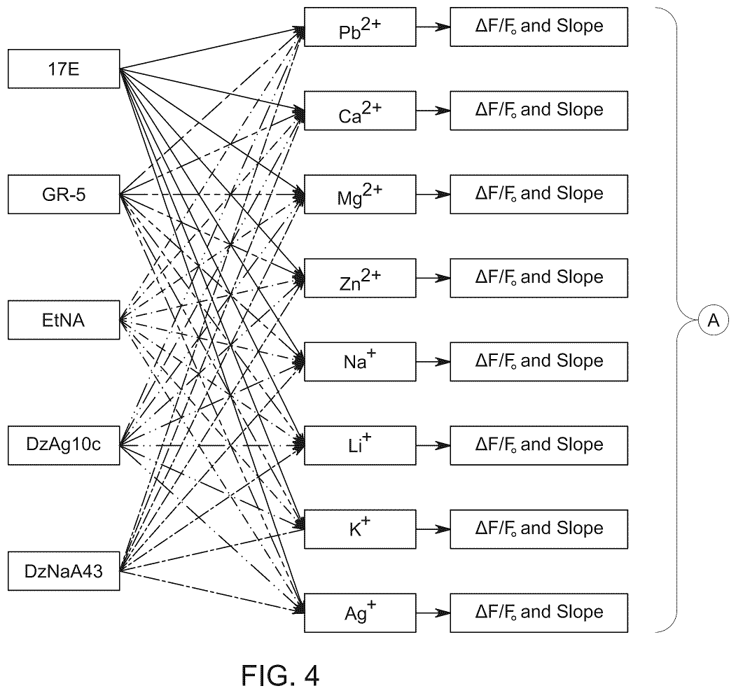

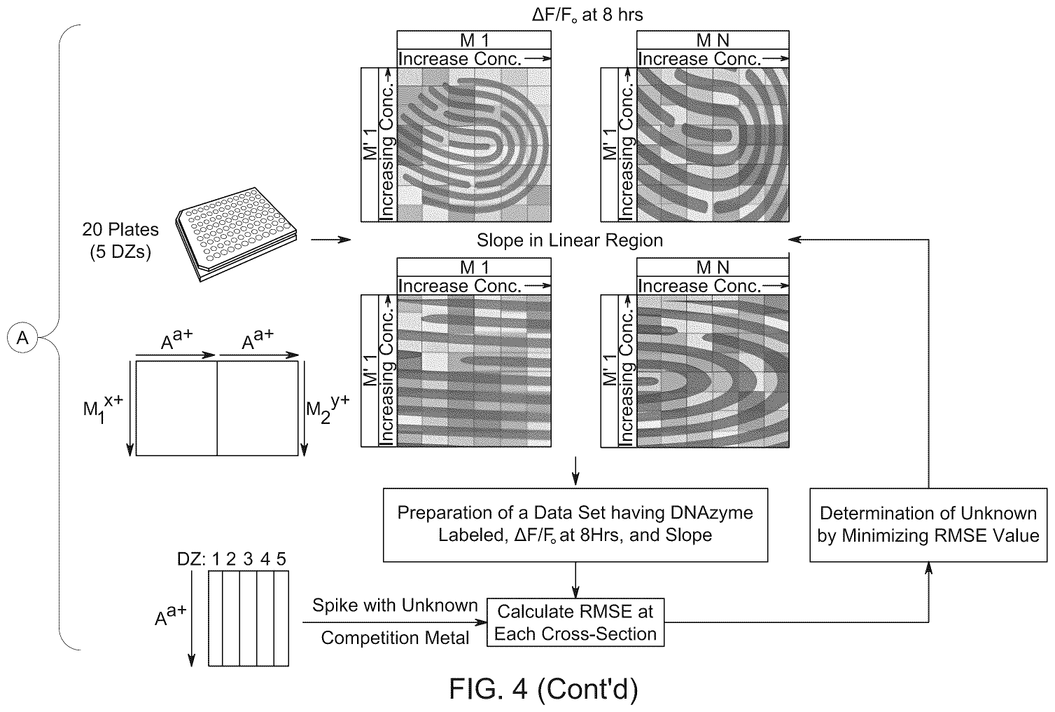

DNAzyme metal ion cross reactivity was measured through the following set-up: For each DNAzyme, 4 black round bottom 96-well plates were used. Each plate was organized into two halves, where the left half corresponded to one competing metal cation and the right half corresponded to another competing metal cation, FIG. 4. All experiments were conducted using the same reaction conditions except varying metal ion concentrations: 50 mM HEPES (pH 7.5, 28 mM NaOH), 100 nM reporter substrate, 25 nM DNAzyme, corresponding native metal, corresponding competing metal, and diluting to a final volume of 100 μL using nanopure water. The native metal ion cofactor concentration increased going across the columns left to right. For Pb²⁺, 0 μM, 0.01 μM, 0.1 μM, 1 μM, 10 μM, and 100 μM concentrations were tested. For Ca²⁺, Ag, and Na, 0 mM, 0.01 mM, 0.1 mM, 1 mM, 10 mM, and 100 mM concentrations were tested. The competing cation, Li⁺, Na⁺, K⁺, Ag⁺, Mg²⁺, Ca²⁺, Zn²⁺, and Pb²⁺ increased in concentration going down the rows. For all competing ions, 0 μM, 0.2 μM, 2 μM, 20 μM, 200 μM, 2 mM, 20 mM, and 200 mM concentrations were tested. The order of reagent addition was water→buffer (10 μL)→substrate (1 μL)→native metal→competing metal→and lastly DNAzyme (1 μL) to initiate the reaction.

### Validation Testing

Metal salt solutions: 2 mM Pb(NO₃)₂, 200 mM Pb(NO₃)₂, 200 μM AgNO₃, and 100 μM Zn(Pb(NO₃)₂ were screened using the DNAzyme array by measuring DNAzyme activity in the presence of the known salt solutions and varying amounts of native metal cofactor, and then comparing the resulting slopes and endpoint measurements (ΔF/F₀ at t=8 hr) to the calibration set. All reactions were conducted using the same reaction conditions except varying the native metal ion concentrations: 50 mM HEPES (pH 7.5, 28 mM NaOH), 100 nM reporter substrate, 25 nM DNAzyme, corresponding native metal, test solution, and diluting to a final volume of 100 μL using nanopure water. Each DNAzyme and metal cofactor concentration (Pb²⁺: 0 μM, 0.01 μM, 0.1 μM, 1 μM, 10 μM, and 100 μM; Ca²⁺, Ag⁺, and Na⁺: 0 mM, 0.01 mM, 0.1 mM, 1 mM, 10 mM, and 100 mM) comprised a single sensor unit in the array, which the array totaled to thirty sensing units. The 96-well plate DNAzyme array was organized so that each column corresponded to a DNAzyme, and proceeding down the rows were increasing native metal cofactor concentration, FIG. 4.

### Data Processing

The initial slope (t=0 through 15 mins) and the endpoint fluorescence (ΔF/F₀ at 1=8 hrs), were obtained from kinetic plots using a python script developed in house, where the initial slope is indicative of the initial reaction velocity. Heat maps for both slope and endpoint fluorescence data were generated in Excel using conditional formatting, where red corresponded to the lowest value observed for a particular feature (either slope or endpoint fluorescence), green corresponded to the highest value for that same feature, yellow corresponded to the 50th percentile, and a gradient in between. Each set of reactions for a single DNAzyme were conditionally formatted independently from the other DNAzymes.

For cluster analysis, endpoint fluorescence, slope, and native metal concentrations were compiled and analyzed using principal component analysis (PCA) and t-distributed stochastic neighbor embedding (t-SNE) to determine if the data clustered based on the competing metal. For PCA, eight components were pre-defined. For t-SNE, the perplexity parameter was investigated using GridSearchCV cross-validation method, where the goal was to find the perplexity value that minimized the objective score calculated for the t-SNE model. Based on the GridSearchCV analysis, a perplexity value of 95 resulted in a minimal objective score. The t-SNE model was then generated on the dataset so that the data points were color-coded based on competing metal. Subsequently, the t-SNE reduced dataset underwent further examination using a k-means clustering algorithm. The k-means clustering analysis aimed to assess the significance of the clusters from the t-SNE analysis. The elbow method was used to determine the optimal number of clusters for the k-means clustering analysis, which was found to be eight.

Identification and quantification of the unknowns were determined by calculating and comparing the root-mean-squared error (RMSE) of the kinetic parameters (endpoint fluorescence (ΔF/F₀) and slope) of the test solution reactions to the cross-reacted metal from the calibration set, and then selecting the condition which yielded the minimal RMSE for either endpoint ΔF/F₀, slope, or both (Table 2.1). In addition, identification of the metal solutions were also determined using the same t-SNE analysis described previously, where the endpoint fluorescence, slope, and concentration of the native metal were adjoined to the calibration data set.

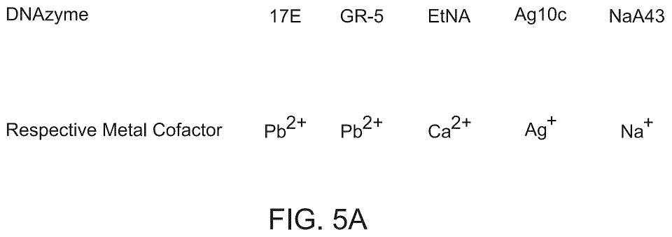

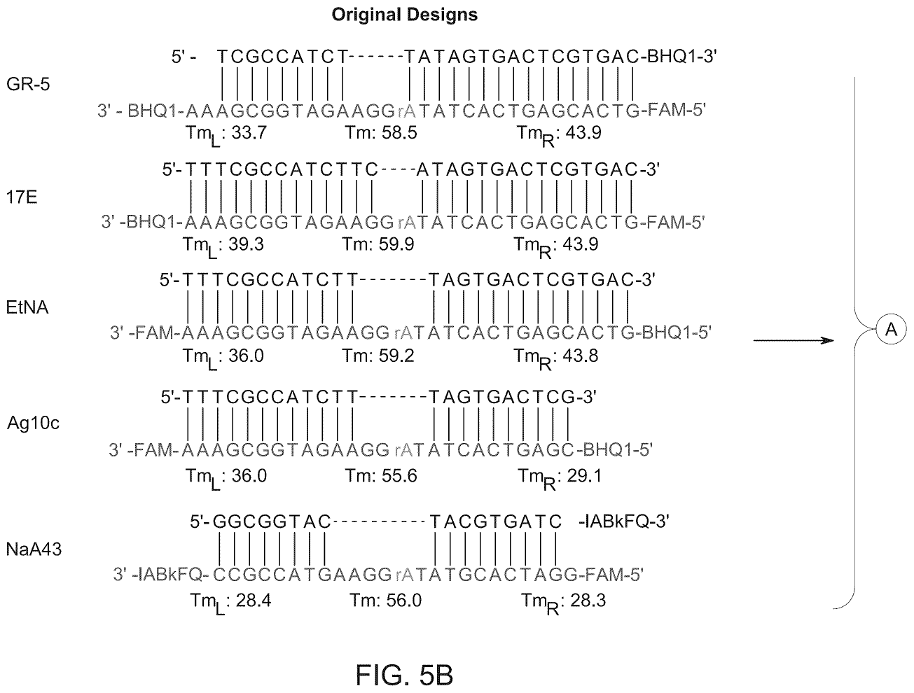

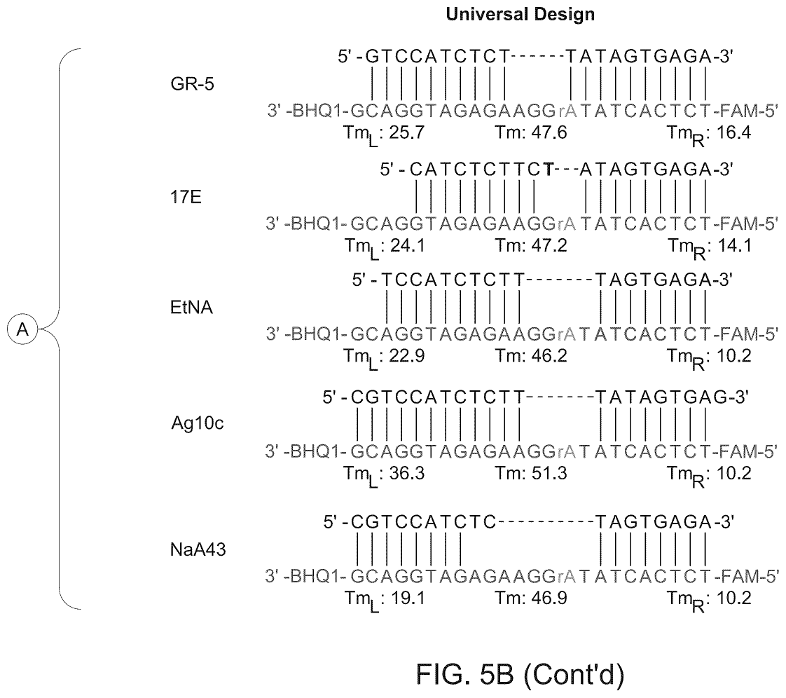

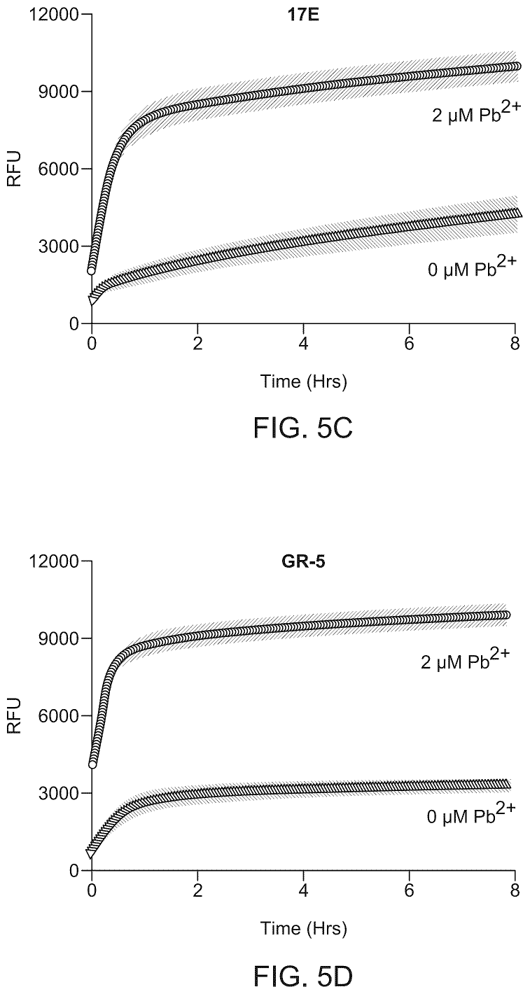

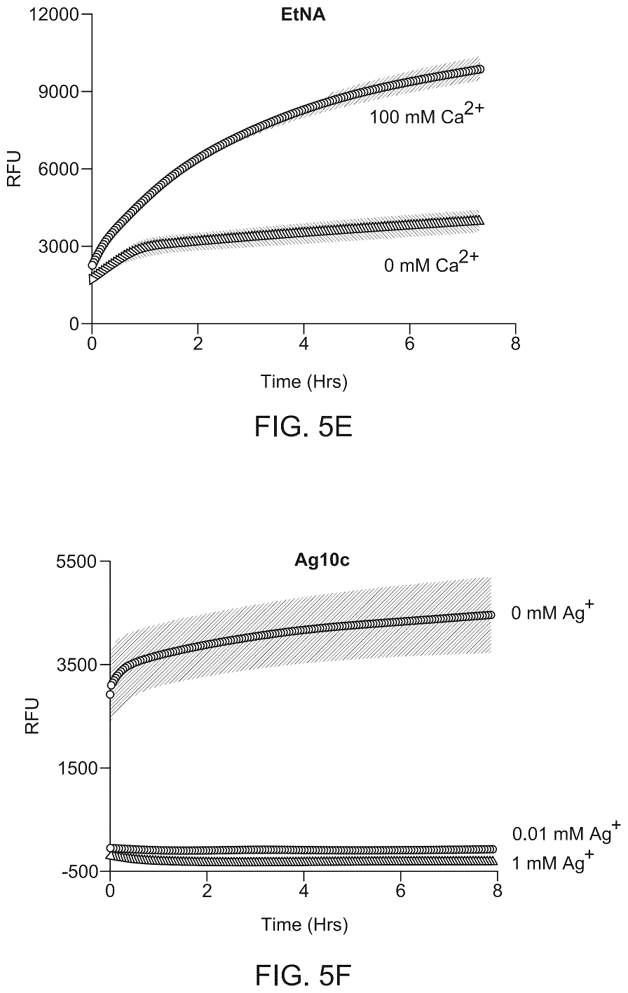

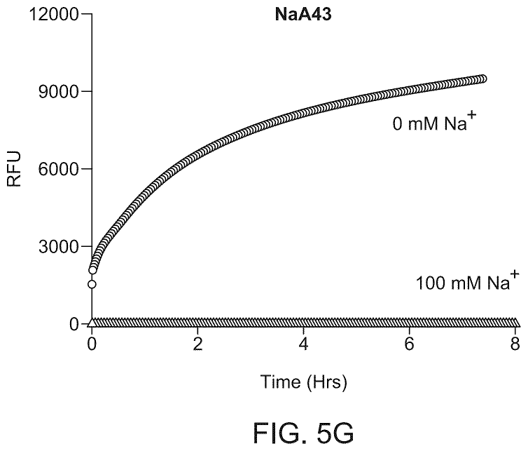

Five DNAzymes, 17E, GR-5, EtNA, Ag10c and NaA43, were selected to comprise the DNAzyme array due to their ability to detect monovalent and divalent metal ions, FIG. 5A. We hypothesized that this diverse metal cofactor selectivity would result in better fingerprinting of metal ions to improve detection accuracy. Moreover, 17E and GR-5 are Pb²⁺-sensitive DNAzymes, and lead is a major environmental pollutant and known to cause devastating neurological defects, particularly in young children. Furthermore, these DNAzymes are all RCDs and cleave at the same ribonucleotide, thus potentially enabling use of a single universal substrate to decrease assay cost.

The universal fluorescent substrate was made using the substrate for GR-5 as the core sequence, comparing the original DNAzyme substrate architectures, and then designing similar melting temperatures for the ‘substrate binding arms’, which are nucleotide sequences flanking the catalytic core and are generally not involved in catalysis. Additionally, the DNAzymes were designed to have the same number of unpaired nucleotides as in their originally reported design. Because NaA43 and EtNA have a larger number of unpaired nucleotides (6 and 4, respectively), additional nucleotides were added to the substrate termini, to maintain similar melting temperatures as the originally reported DNAzyme-substrate pairs, FIG. 5B. Since DNAzyme activity is dependent on the substrate sequence, we tested whether the universal substrate can still be cleaved. Each DNAzyme was independently tested under their respective reported optimized buffer condition, FIG. 5C-G. All DNAzymes except for Ag10c showed an increase in fluorescence in the presence of their native metal cofactor compared to when there was no metal cofactor present. Surprisingly, Ag10c, a silver-sensitive DNAzyme, showed inhibition in the presence of 1 mM Ag. Sometimes, too high a metal concentration can lead to DNAzyme inhibition. Therefore, we proceeded to test 0.01 mM Ag to determine whether activity could be recovered, which was not. However, inhibition occurred to a slightly lesser degree. Despite this, we opted to retain Ag10c in the DNAzyme array, anticipating the possibility of uncovering a distinctive inhibition pattern when testing cross-reactivity with other metals.

### DNAzyme Cross-Reactivity Studies and Clustering Analysis

Upon validating DNAzyme activity for the universal substrate, we measured DNAzyme cross-reactivity with common metal interferents (monovalent cations: Li and K′, and divalent cations: Mg²⁺ and Zn²⁺) with concentrations ranging from 0 μM to 200 mM and spanning 7 orders of magnitude. After carrying out the fluorescence kinetic assays, the endpoint fluorescence, ΔF/F₀ and the initial slope were extracted as our two signal features. To determine if the DNAzyme showed a unique pattern of activity for each competing metal, we conditionally formatted the slope and endpoint fluorescence data, grouped by each DNAzyme (vertical column sets in FIGS. 6A & B, respectively), where red corresponded to the lowest response observed, yellow corresponded to the 50^{th}-percentile, green corresponded to the highest response observed, and a gradient in between.

By initial qualitative observation, it appears that no rows were identical, signifying that each competing metal produces a distinguishable signal pattern. To investigate this further, we performed principal component analysis (PCA) on the data. However, PCA was unable to fully separate the clusters based on competing metal. In addition, there was a strong grouping of data from each competing metal label along the axial lines y=x and y=−x, which is indicative of strong non-linear pattern. Thus, we resolved to find another dimension reduction technique that was non-linear and could best separate the data labeled by competing metal. As a result, we proceeded to test whether a non-linear dimension reduction technique (t-SNE) could separate the data based on competing metal, FIG. 6C. Through t-SNE analysis using a perplexity value of 95, multiple centroid clusters corresponding to different competing metals emerged with little overlap of the clusters. However, a region comprised of clusters having minimal separation appeared in the center. Upon closer inspection of the data, this group of clusters corresponded to very dilute metal concentrations, which would be expected to exhibit little to no distinguishable DNAzyme activity. Nonetheless, there appeared to be a correlation in the data, so we carried out k-means clustering to see if the clusters were significantly separated, FIG. 6D. In doing this, we observed distinct clusters for each of the competing metal ions: Li⁺, Na⁺, K⁺, Ag⁺, Mg²⁺, Ca²⁺, Zn²⁺, and Pb²⁺ at higher metal concentrations, greater than 200 μM. The metal concentrations less than 200 μM, bifurcated into two congruency classes by k-means clustering (FIG. 6, Cluster 1 and Cluster 8). This shows that this region does consist of unique features, but the features are not significant enough to fully separate out the data points based on identity of the competing metal. Together, this shows that metal concentrations greater than 200 μM can be quantified through t-SNE analysis.

### Testing Array Accuracy

The array was validated using known control solutions containing 2 mM Pb(NO₃)₂, 200 mM Pb(NO₃)₂, 200 μM AgNO₃, and 100 μM Zn(NO₃)₂. These validation solutions were chosen as they are conditions known to produce false positive results using a single DNAzyme test approach. For example, Zn²⁺ is a major interferent for lead sensitive DNAzymes. In addition, the inclusion of 100 μM Zn²⁺ aimed to test the array's ability to identify the correct metal for a metal concentration that was not specifically tested in the calibration set. In addition, we included 200 μM Ag⁺ to investigate the detection capability of the array for silver, given that both 10 μM and 1 mM Ag⁺ inhibited Ag10c activity in the preliminary studies.

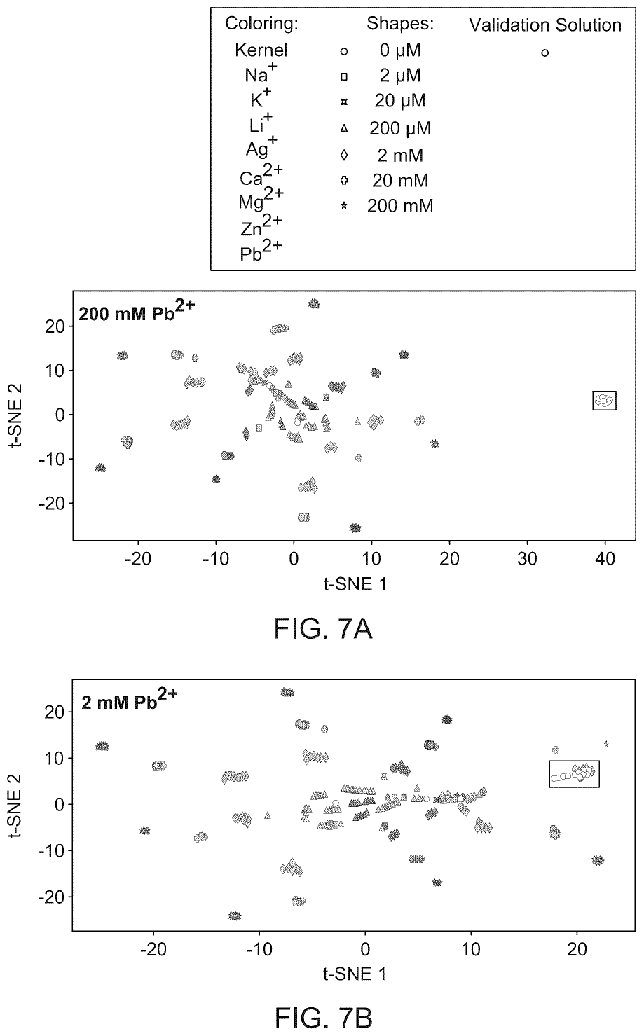

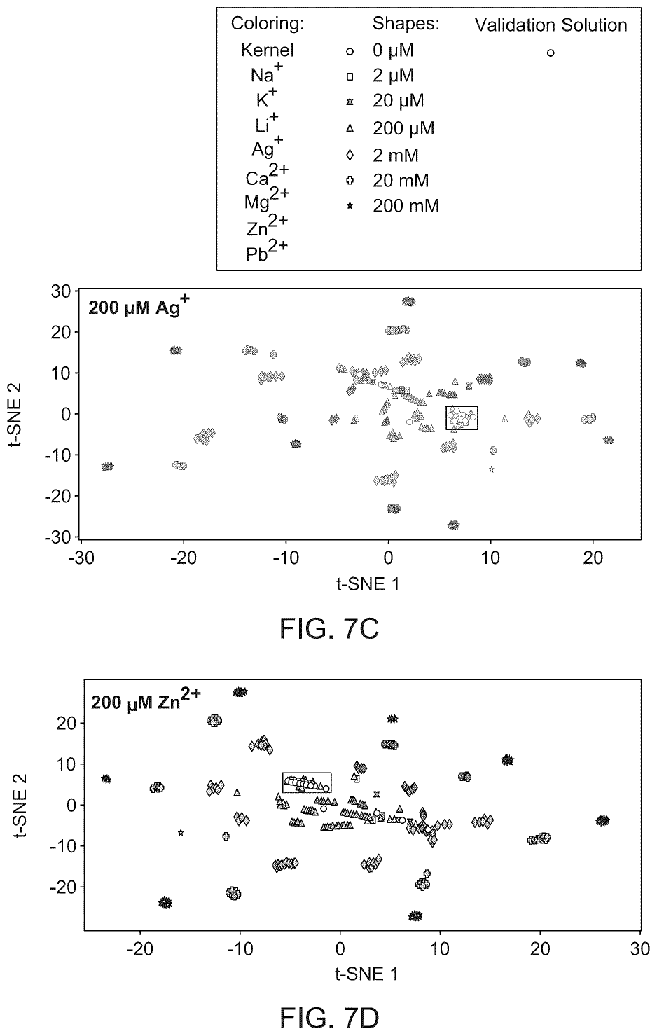

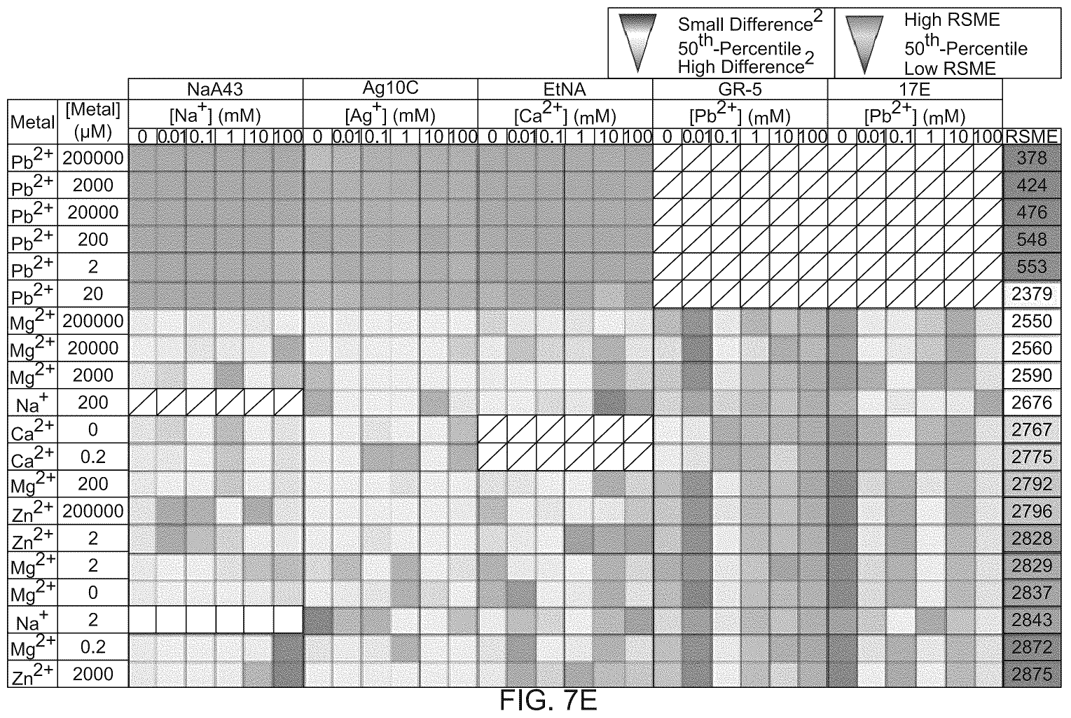

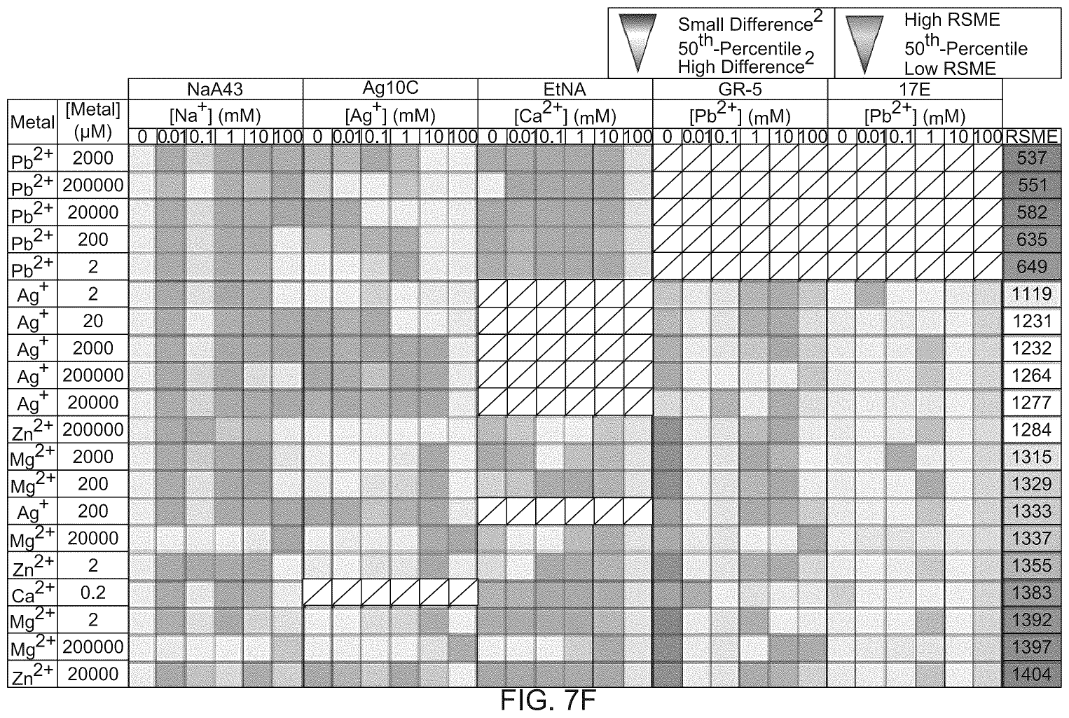

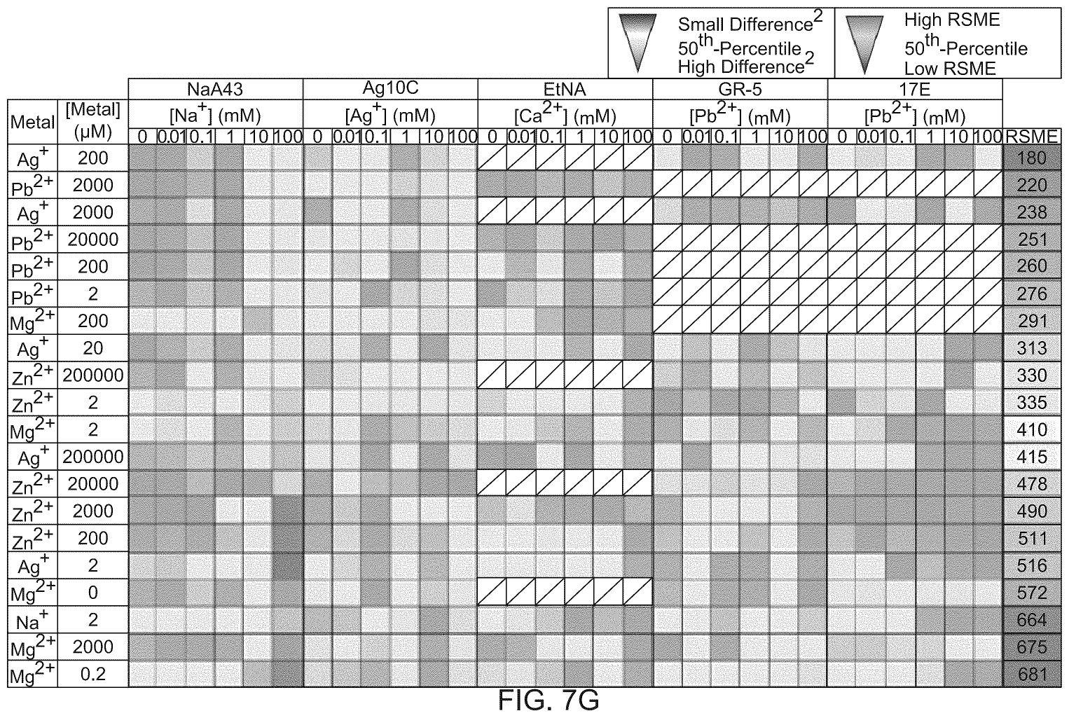

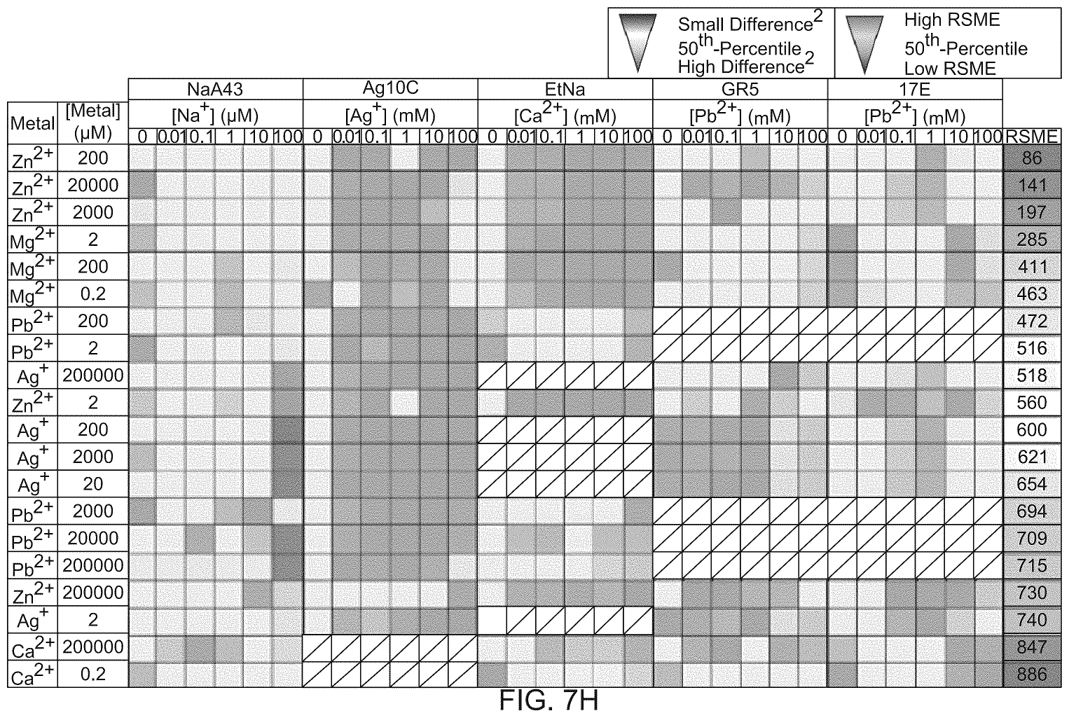

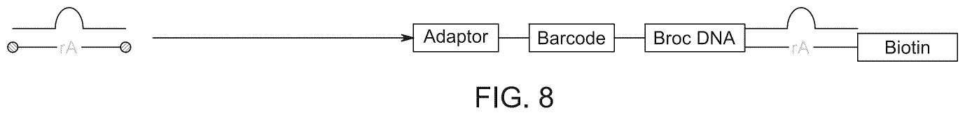

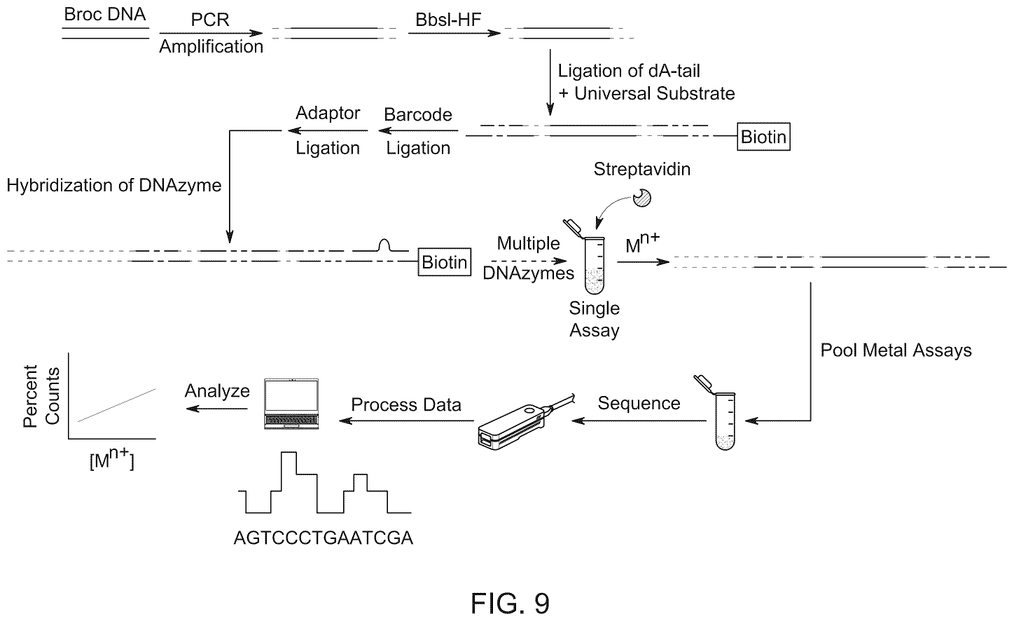

Initially, the quantitative patterned response of the array was assessed through t-SNE analysis by examining how the test solutions clustered with the calibration set. Remarkably, t-SNE analysis successfully grouped all four test solutions with their respective metal (FIG. 7A-D) and correctly quantified concentrations for 2 mM Pb²⁺, 200 mM Pb²⁺ and 200 μM Ag⁺, where 100 μM Zn²⁺ grouped with 200 μM Zn²⁺ cluster. The array's ability to quantitatively determine the solution metal concentration was also assessed by computing and comparing the root-mean-squared error (RMSE) of the kinetic parameters (endpoint ΔF/F₀, slope, or both) from reactions involving the validation solutions to those from the cross-reacted metal in the calibration set. The condition yielding the minimal RMSE was chosen and summarized in Table 1. Notably, when only using the slope signal feature FIG. 7E-H, the same three metal solutions were successfully identified, where the 100 μM Zn²⁺ had the lowest RMSE for 200 μM Zn²⁺, which was the closest concentration in the calibration set. Similarly, when calculating the minimal RMSE using both endpoint ΔF/F₀ and slope, we were able to correctly identify the metal ion in all four validation solutions as well. In contrast, when calculating the minimal RMSE only using endpoint ΔF/F₀ data, only the 100 μM Zn²⁺ solution was correctly identified, which it was identified as 200 μM Zn²⁺. The 2 mM Pb²⁺, 200 mM Pb²⁺, and 200 μM Ag⁺ solutions were identified as 200 μM Na⁺, 2 μM Na⁺, and 200 μM Na⁺, respectively. The top twenty closest hits are summarized in the conditionally formatted RMSE data. We believe this poor performance in using endpoint fluorescence as a signal feature is due to the assay being designed as multi-turnover, where higher DNAzyme activity cannot be distinguished after reaching saturation. Therefore, we determined that using the slope parameter as the signal feature is the most accurate way to analyze the array.

TABLE 1

| | | Initial | Endpoint ΔF/F₀ and | |
|---|---|---|---|---|
| Validation | Endpoint | Reaction | Initial Reaction | |
| Solution | ΔF/F₀ | Velocity | Velocity | t-SNE |
| 2 mM Pb²⁺ | 200 μM Na⁺ | 2 mM Pb²⁺ | 2 mM Pb²⁺ | 2 mM Pb²⁺ |
| 200 mM Pb²⁺ | 2 μM Na⁺ | 200 mM Pb²⁺ | 200 mM Pb²⁺ | 200 mM Pb²⁺ |
| 200 μM Ag⁺ | 200 μM Na⁺ | 200 μM Ag⁺ | 200 μM Ag⁺ | 200 μM Ag⁺ |
| 100 μM Zn²⁺ | 200 μM Zn²⁺ | 200 μM Zn²⁺ | 200 μM Zn²⁺ | 200 μM Zn²⁺ |

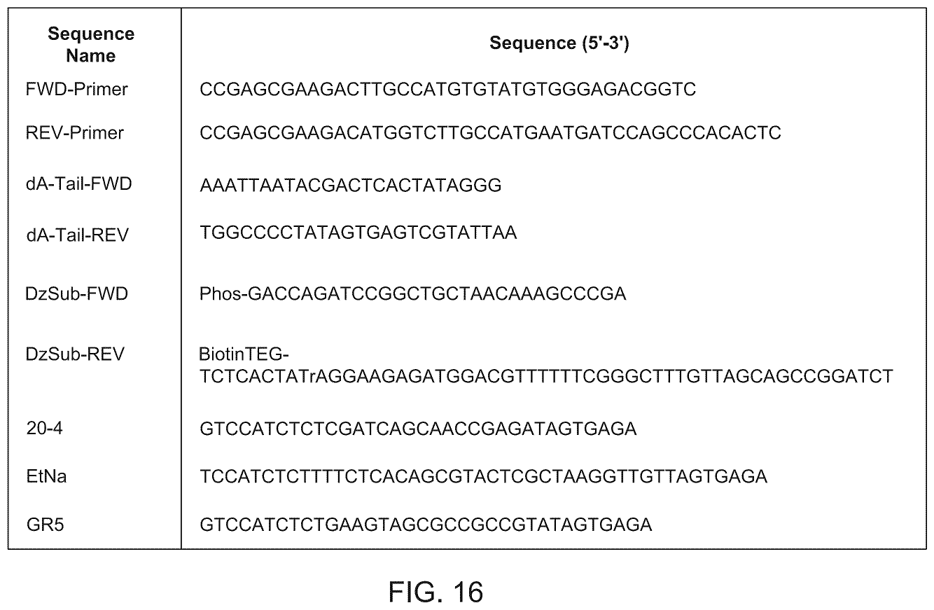

Materials: All oligonucleotides were purchased from IDT (Newark, USA). Sequences are summarized in FIG. 16. The pET28c-F30-2xdBroccoli (Broc) plasmid was purchased from Addgene (plasmid #66843; http://n2t.net/addgene: 66843; RRID: Addgene_66843). Lead (II) nitrate (ACS certified), lithium nitrate (99.99% trace metals basis), and HEPES (BioPerformance Certified, ≥99.5%) were purchased from Sigma-Aldrich. Calcium nitrate tetrahydrate (ACS Certified) was purchased from Fisher Chemicals. Sodium nitrate (ACS certified) was purchased from Alfa Aesar. Sodium hydroxide was purchased from Fisher BioReagents. All sequencing experiments were carried out on a MinION Mk1B nanopore sequencer. The Flongle Flow Cell Adaptor, flongle flow cells (R10.4.1), and the Native™ barcoding kit 24 v14 (Product #: SQK-NBD114.24) available from Oxford Nanopore. Streptavidin beads, BbsI-HF, T4 ligase, and quick ligase purchased from NEB. Kapa HiFi was purchased from Roche Sequencing Solutions.BD Difco Dedhydrated Culture Media: Luria-Bertani Miller Broth, and black round bottom 96-well plates (Product #: 15-100-175) were purchased from Fisher Scientific. Kanamycin monosulfate (USP Grade) was purchased from Gold Biotechnology Inc. DNA purification spin columns, Qiagen MiniPrep Kit and Qiagen PCR Cleanup Kit were purchased from Qiagen.

### DNAzyme Design and Manufacture

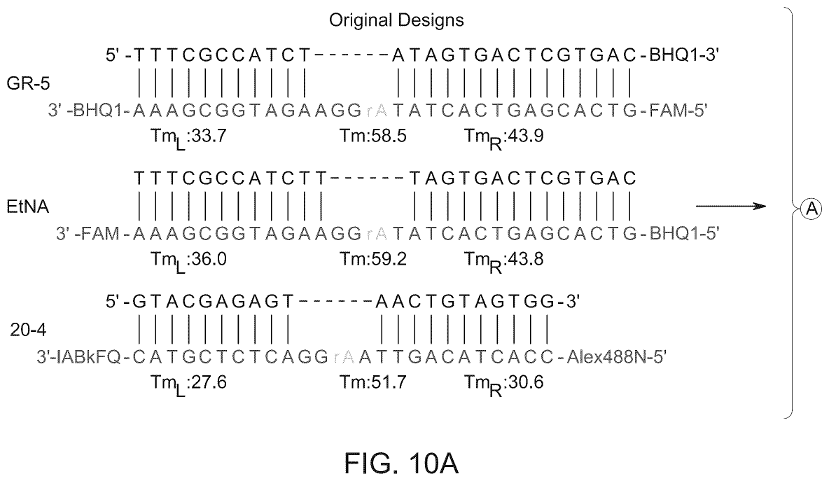

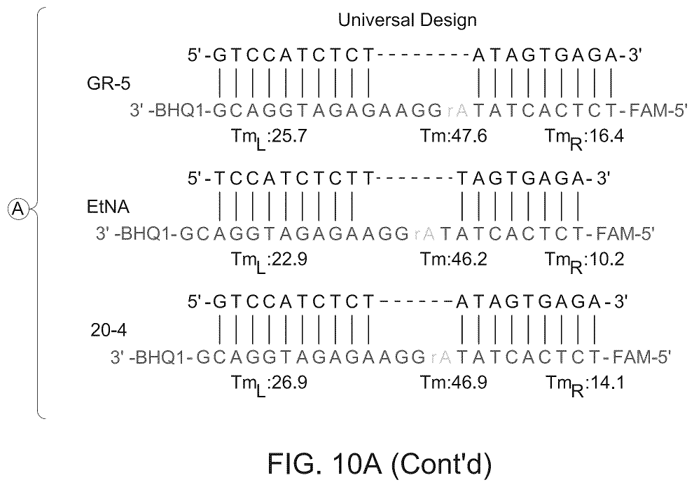

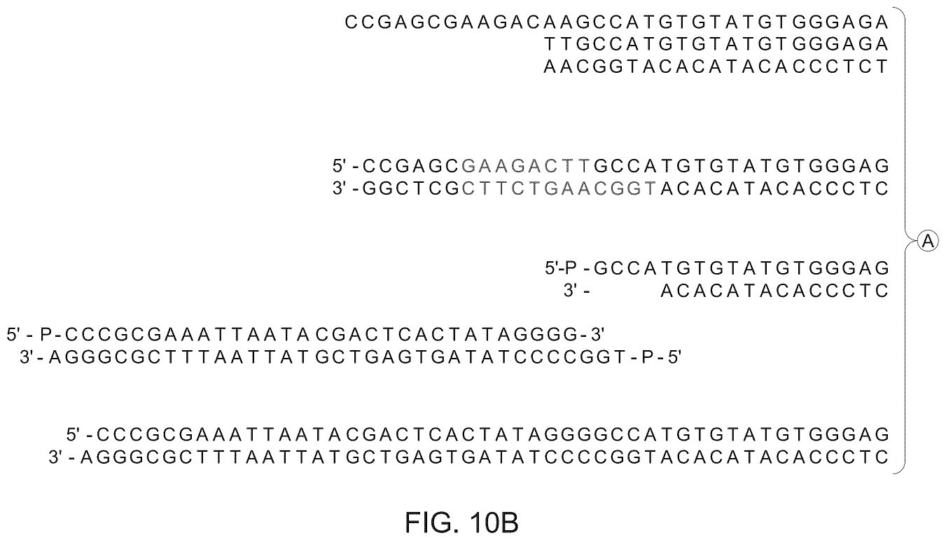

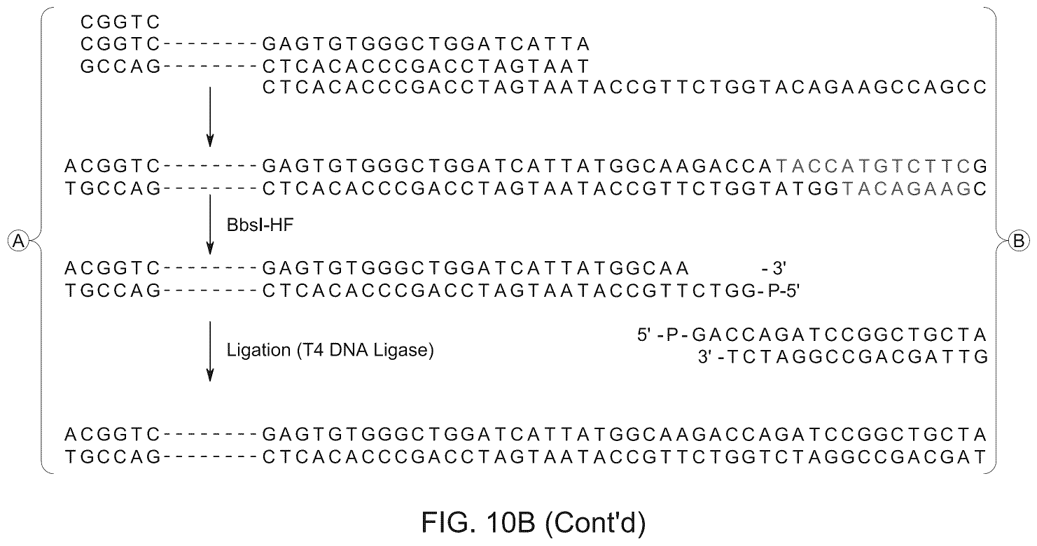

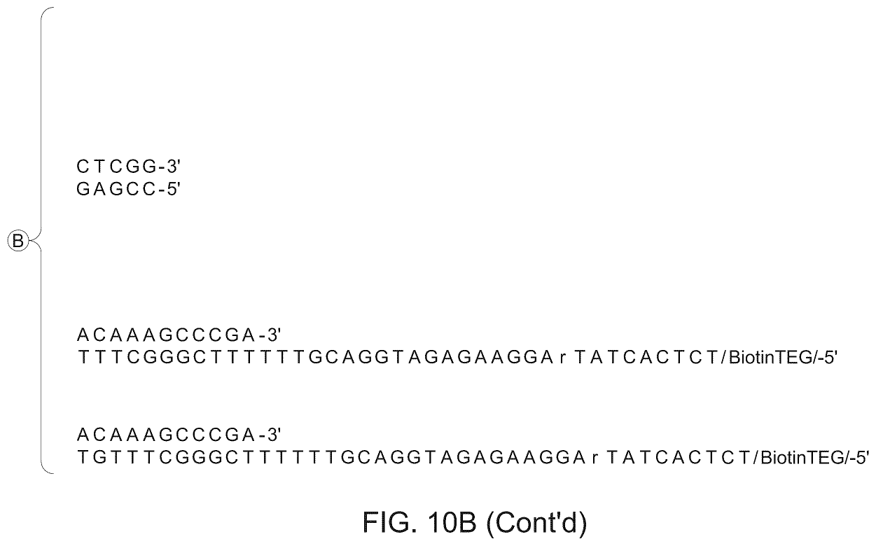

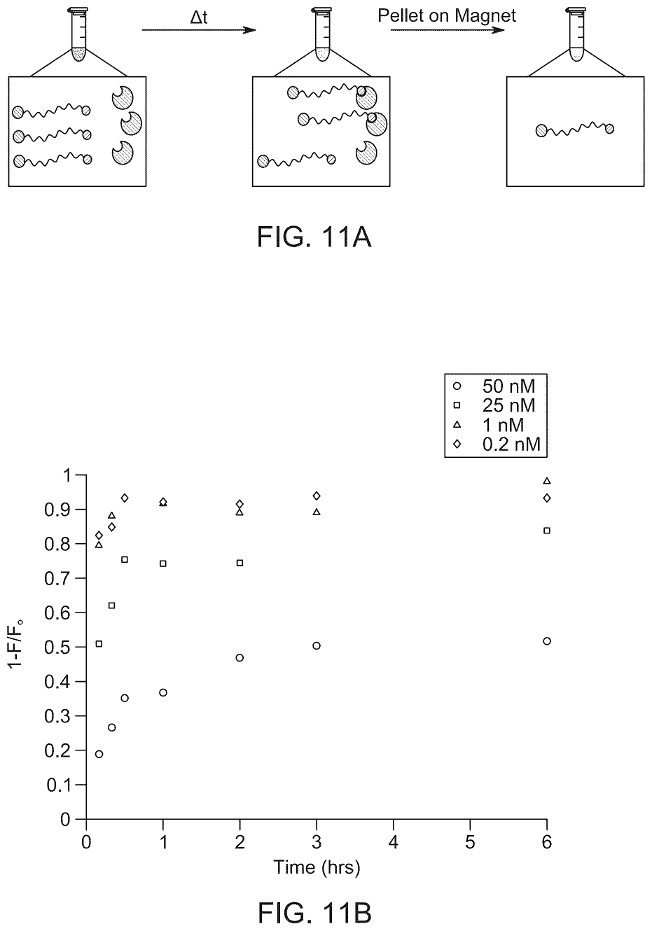

The DzNanoporeSeq substrate DNA was assembled using a type IIS ligation strategy, summarized in FIG. 10 and, where barcoded nanopore adaptors and the DNAzyme substrate were selectively ligated to a ˜200 bp amplicon from Broc Plasmid DNA. Broc Plasmid DNA was isolated from an overnight culture (LB supplemented with 50 μg/mL of kanamycin) and purified using the Qiagen MiniPrep Kit. This amplicon DNA was used to increase sensor length ˜200 bp and was conveniently available.

Type IIS restriction ligation cloning was used to selectively ligate the nanopore adaptor and DNAzyme substrate onto the 200 bp amplicon. Specifically, forward and reverse primers encoding for BbsI-HF restriction sites were used to amplify a 200 bp region of the plasmid. The PCR amplified product was purified using the Qiagen PCR cleanup kit, and DNA was quantified using nanodrop. Next, 10 pmol of the amplicon was restriction-digested using 40 units of NEB BbsI-HF, with an incubation time of 1 hr at 37° C. The restriction-digested product was purified using Qiagen PCR cleanup kit. Simultaneous ligation of the DNAzyme substrate and a dA tail duplex were selectively ligated onto the termini of the digested amplicon through the resulting custom four nucleotide overhangs from BbsI-HF digestion. Briefly, 8 pmol of digested DNA, 32 pmol of dA tail duplex containing a sticky end overhang at opposite terminus of the dA tail, and 32 pmol of DNAzyme substrate were mixed together and ligated using 800 units of T4 ligase in a total reaction volume of 40 μL in 1× ligase buffer at 16° C. for 16 hrs, which was followed by heat inactivation at 65° C. for 10 mins. The DNA ratios were 1:4:4 for amplicon, dA tail duplex, and DNAzyme substrate, respectively. The ligated product was then purified using gel extraction with a 2% agarose gel.

Nanopore barcodes were then ligated to the dA terminus of the DNA using Native barcoding NB24 v14 and corresponding nanopore protocol. Those of ordinary skill in the art will recognize that conventional means of ligation may be interchanged with the dA tail ligation strategy described herein and still fall within the general inventive concepts. Briefly, 200 fmol of the of the DNA was ligated to 20 barcodes using 400 units of T4 ligase with 1× ligase buffer in a total reaction volume of 40 μL at 25° C. for 20 mins for each barcode. The ligation reaction was stopped through the addition of EDTA provided in the nanopore Native barcoding kit. The resulting ligated product was then purified using 1.8× AmpureXP beads and eluted with autoclave water. The nanopore adaptor was then ligated onto the barcoded DNA using the nanopore protocol. Briefly, all of the eluted barcoded DNA was ligated with the nanopore adaptor using 1 μL of quick ligase in a total reaction volume of 70 μL and 1× quick ligase buffer at 25° C. for 20 mins. Following this, 1.8× AmpureXP Beads was added to the ligation reaction, the beads were washed with the short fragment buffer, and the DNA was eluted using the supplied elution buffer.

The DNAzyme sensors were then hybridized to the barcoded substrate containing the Native adaptor. The DNAzymes was hybridized as follows for 18 of the 20 barcodes, since two barcodes are reserved for threshold testing: 200 fmol of the adaptor ligated DNA was hybridized to 200 fmol of corresponding DNAzyme in 1×SSC buffer in a total reaction volume of 40 μL. For the other two hybridization reactions, 20 fmol of adaptor ligated DNA was hybridized to 20 fmol of corresponding DNAzyme using 1×SSC buffer in a total reaction volume of 20 μL. Then the hybridized DNA sensors, 20 fmol each, were pooled together. Next, the pooled DNAzymes were incubated with 1 μg of streptavidin beads (SA) in 50 mM HEPES (32 mM NaOH, pH 7.5) and 100 mM of NaNO₃ (reaction buffer) in a total volume of 90 μL for 3 hrs at 25° C. with shaking at 1400 rpm. The two barcoded DNAzyme sensors hybridized at 20 fmol were not pulled together but were incubated with 0.33 μg SA in the reaction buffer having a total volume of 90 μL for 3 hrs at 25° C. with shaking at 1400 rpm.

### Biotin Binding Studies

The optimal binding time between the biotin-labeled universal substrate and biotin was measured using a fluorescent assay. Briefly a biotinylated fluorescent DNA oligonucleotide was mixed with SA beads at varying concentrations of DNA, which were then incubated for varying amounts of time. Then the loss of fluorescence intensity of the supernatant as a function of time using a BioTek Synergy LX plate reader equipped with a tungsten lamp, a green filter (Ex. 485/20, Em. 528/20) and a gain of 30.

To accomplish this, SA beads were prepared by first pelleting 10 μg of SA beads on a magnet until the solution was clear. The supernatant was removed, and the beads were washed three times with 10× volume of streptavidin wash buffer (20 mM Tris, 15 mM HCl, pH 7.5, 0.5 M NaCl, and 1 mM EDTA) by pelleting on magnetic removal of supernatant each time. The beads were then suspended in 10 μL of nuclease free water. The concentrations of the biotin-labeled fluorescent substrate tested were: 0.2 nM, 1 nM, 25 nM, and 50 nM. For each concentration tested, eight reaction vessels were prepped corresponding to the amount of time the biotin magnetic beads were incubated with the streptavidin-labeled universal substrate. The times were: 0 mins, 10 mins, 20 mins, 30 mins, 1 hr, 2 hrs, 3 hrs, and 6 hrs. To each reaction vessel the resuspended streptavidin beads (10 μL), HEPES (50 mM, 32 mM NaOH, pH 7.5; 5 μL), NaNO₃ (5 μL), the biotin-labeled universal substrate (10 μL), and water (20 μL) were added. The order of addition was water→HEPES→NaNO₃→biotin labeled substrate→streptavidin beads to initiate binding. Each reaction vessel was shaken on an Eppendorf F2.0 ThermoMixer shaker at 25° C. and 1400 rpm for its designated amount of time. Then, the vessel was briefly centrifuged and allowed to pellet on a magnet until the solution became clear. The supernatant was removed (˜95 μL) and placed in a 96-well plate. Then, the fluorescence of the supernatant was taken. This procedure was repeated in triplicate for each substrate concentration at each time point. A kinetic scatter plot was made of 1-F/F₀, where F₀ was the fluorescence of the 0 mins sample.

### Metal Testing

Following the incubation of the pooled DNAzymes and SA, 10 μL of target metal was added at the desired final concentration. For Li⁺ and Ca²⁺, the concentrations tested were: 0, 1, 5, 10, 70, and 100 mM. For Pb²⁺, the concentrations tested were: 0, 10, 50, 100, 500, and 1000 nM. The spiked samples were then incubated at 25° C. for 2 hrs with shaking at 1400 rpm. Following the incubation, each reaction vessel was briefly spun down and pelleted on a magnet until the supernatant was clear. Then all the supernatants were pooled together and combined with 1.8× AmpureXP beads. The beads were incubated at 25° C. for 10 mins with shaking at 1400 rpm. The beads were then briefly spun down and pelleted on a magnet until the supernatant was clear. The supernatant was removed, and the beads were then washed with 125 μL of short fragment buffer and allowed to pellet on a magnet until supernatant was clear. The supernatant was removed. This process was repeated for a second time. The beads were then mixed with 15 μL of elution buffer supplied by the nanopore kit and were incubated at 37° C. for 10 mins with shaking at 1400 rpm. The beads were then briefly spun down and allowed to pellet on a magnet. The supernatant was then saved for use in the sequencing experiment.

### DNAzyme Kinetic Studies

DNAzyme catalytic activity was determined by measuring the rate of hydrolysis of a DNA/RNA chimera substrate that was functionalized with a 5′ 6-fluorescein (FAM6) and a 3′ black hole quencher (BHQ). Hydrolysis was measured by monitoring fluorescence intensity as a function of time using a BioTek Synergy LX plate reader equipped with a tungsten lamp. Fluorescence measurements were taken every minute for two hours at room temperature (24° C.) using a green filter (Ex. 485/20, Em. 528/20) and a gain of 50. In between measurements, plates were shaken with double orbital rotation.

DNAzyme metal ion cross-reactivity was measured with the following setup: initially, each DNAzyme was hybridized individually to a uniquely barcoded universal substrate (1:1) using SSC buffer to have a final concentration of 250 nM. The concentrations of Li⁺ and Ca²⁺: tested were: 0, 1, 5, 10, 70, and 100 mM. For Pb²⁺ the concentrations tested were: 0, 10, 50, 100, 500, and 1000 nM. To a 96-well plate, 10 μL of HEPES (50 mM, 32 mM NaOH, pH 7.5), 10 μL of NaNO₃ (100 mM) stock, 10 μL of DNAzyme: Universal Substate duplex (25 nM), 10 μL of metal were added together. Then sufficient water was added to bring the final reaction volume to 100 μL. The order of addition to the well was water→HEPES→NaNO₃→DNAzyme: Universal Substrate→metal to start the reaction.

### Nanopore Sequencing

For the calibration experiments, known mixtures of barcoded DNA were formulated and sequenced using the MinION sequencing device. Briefly, four formulations were tested and individually sequenced where the total amount of DNA and dynamic range were altered. For the 19.3 fmol formulation, the different amounts of barcoded DNA present were 0.05, 0.5, 1.25, 2.5, 5, and 10 fmol, where each amount had a unique barcode. For the 38.875 fmol formulation, the different amounts of barcoded DNA present were 0.125, 1.25, 2.5, 5, 10, and 20 fmol, where each amount had a unique barcode. For the 57.9 fmol formulation, the different amounts of DNA present were 0.15, 1.5, 3.75, 7.5, 15, and 30 fmol, where each amount had a unique barcode. For the 185.5 fmol formulation, the different amounts of barcoded DNA present were 21, 25, 29, 33, 37.5, and 40 fmol, where each amount had a unique barcode. Once the formulation was made, 12 μL the formulation was combined with 37.5 μL sequencing buffer and 25.5 μL library beads for a total volume of 75 μL. The sequencing mixture was then loaded to the flow cell and sequencing was carried out with a sequence time of 12 hrs and the minimum sequence length set to 20 bp and live basecalling was turned off.

For heavy metal detection studies, the purified cleaved DNAzyme products ascertained from pooling the individual reactions on SA beads together (˜12 μL) were combined 37.5 μL sequencing buffer and 25.5 μL library beads for a total volume of 75 μL. The sequencing mixture was then loaded to the flow cell and sequencing was carried out with a sequence time of 12 hrs and the minimum sequence length set to 20 bp and live basecalling was turned off.

### Analysis of Sequencing Data

The data collected from the sequencing run was analyzed using Dorado version 0.5.2, where the DNA was first base called using the hac model and dorado basecaller command, then the barcodes were called, and finally the DNA was aligned to the reference substrate sequence using the dorado aligner command. The barcode summary was then analyzed using an in-house Python script to count the number of occurrences of each barcode. Then a scatter plot for each sequencing experiment was generated of the number of counts versus the amount of DNA loaded. A linear line-of-best fit was generated.

### DNAzyme Sensor Design

Traditional DNAzyme sensors function where substrate hydrolysis results in a signal response. For example, for a fluorescence readout, the reporter substrate is functionalized with a fluorophore and quencher, where hydrolysis results in the fluorophore being separated from the quencher, and thus an increase in fluorescence.

Since nanopore sequencing is label free, the substrate strand no longer needs to be fluorescently labeled because the cleaved product is shorter compared to the intact substrate, which can be distinguished by the MinION. The Dz-substrate complex only requires having a nanopore adaptor so that the helicase can selectively ratchet the barcoded substrate through the pore, where the barcode encodes for the corresponding CNA sensor. Since both the product and substrate can translocate through the pore, a biotin was functionalized to the substrate terminus, so that unhydrolyzed DNA can be captured and removed by streptavidin (SA) coated microparticles. This will enable detecting only cleaved product and improving assay sensitivity. The substrate design compared to fluorescent CNA sensors is summarized in FIG. 10A.

For initial proof-of-concept studies highlighting the multiplex capabilities, DNAzymes 20-4, EtNa, and GR-5 were investigated. These DNAzymes were chosen due to their diverse range in metal cofactors detected, which are, Li⁺, Ca²⁺, and Pb²⁺, respectively. To improve the substrate compatibility with Oxford Nanopore sequencing, a DNA fragment with minimum length of 200 bp was incorporated into the designs, as the sequencer was not able to detect DNA fragments less than 200 bp efficiently because shorter DNA fragments transport too fast through the pore. This fragment was chosen as Broc, a plasmid encoded gene that encodes for the RNA aptamer broccoli, since this was readily available from the lab and is ˜200 bp long (234 bp).

A key design feature for DzNanoporeSeq sensors is selective functionalization at each terminus with a duplex encoding a dA-tail with the sequencer adapter and a biotinylated DNAzyme substrate, respectively. The dA-tail is required for ligating on the Oxford Nanopore Native barcode. As a result, a type IIS restriction-digestion and ligation strategy was developed, where PCR extension to incorporation BbsI-HF cut sites on 5′ and 3′ termini of the Broc DNA sequence. In doing this, the unique overhangs that are ascertained from the restriction-digestion reaction of the amplicon with BbsI-HF allowed for the site-selective ligation of the dA-tail duplex onto the 5′ terminus of the amplicon and the site-selective ligation of the DNAzyme universal substrate onto the 3′ terminus of the amplicon.

Gel electrophoresis was carried out to determine if the ligation strategy of adjoining the dA-tail duplex and the DNAzyme universal substrate was successful. The restriction-digestion appeared to be successful since the restriction-digested product (lane 3) seemed to be smaller size compared to the PCR amplicon (lane 2). A size decrease of 263 to 222 bp was expected. The gel shift is very small, so the result is somewhat ambiguous. Since the subsequent ligation steps require that the restriction digestion to be successful, we continued sensor assembly because successful ligation would be indicative of successful restriction digestion. Next, sequential ligations and a double ligation were tested and assessed. Individual ligation of the dA-tailed duplex (left part of the sensor) seemed successful as a slight increase in size was observed. The anticipated size increase was expected to be 247 bp. Then the universal substrate containing the biotin was ligated onto the dA-tailed amplicon. This ligation also seemed successful as a large gel shift for the ligated product was observed. A size increase of 247 to 276 bp was expected. Lastly, the double ligation seemed to be successful since an increase in size was observed compared to the restriction-digested product and had approximately the same size as the final sequentially ligated product. Although the double ligation is lower than the expected 276 bp, it is still slightly larger than the dA-tailed duplex ligated product (lane 3). This result suggested that the double ligation was successful, and that the slightly lower band position than expected was most likely due to a long single-stranded DNA overhang.

Further, since the ultimate signal readout is sequencing, the ligation efficiency can be quantified through sequence analysis. Additionally, sequencing can differentiate unreacted sensors that were not fully removed prior to sequencing. To this end the sequences that were ascertained from the heavy metal studies were aligned to the substrate sequence. For both nanopore sequencing Li experiments, about half of the sequences ascertained have the cleaved substrate sequence present, where the other half had uncleaved substrate. No sequences were observed having no substrate altogether (cleaved or uncleaved), which shows the ligation was very efficient. Likewise, both nanopore sequencing Ca²⁺ experiments and Pb²⁺, about half of the sequences ascertained have the cleaved substrate sequence present. Since no sequences were observed having no substrate, we can estimate that the ligation strategy was ˜100% successful.

### Biotin Binding Studies

To determine the optimal amount of time for binding of the biotinylated substrate DNA to streptavidin magnetic beads, a fluorescence kinetic assay was performed using biotinylated and fluorescently labeled DNA. Since a low sensor amount of 60 fmol (0.6 nM for our assay) is required for nanopore sequencing, a concentration dependence was carried out. Results show, regardless of DNA concentration, all samples had optimal streptavidin binding after 2 hrs. Furthermore, as the DNA concentration decreased, there was an overall trend for increased streptavidin binding (93% for 0.2 nM) and faster binding kinetics. We speculate this is due to sterics and electrostatic repulsion at higher concentrations. Ultimately, these results show there is efficient DNA binding at concentrations less than 25 nM, which is the optimal concentration for nanopore sequencing.

Investigation into Parameters Important for Quantitation

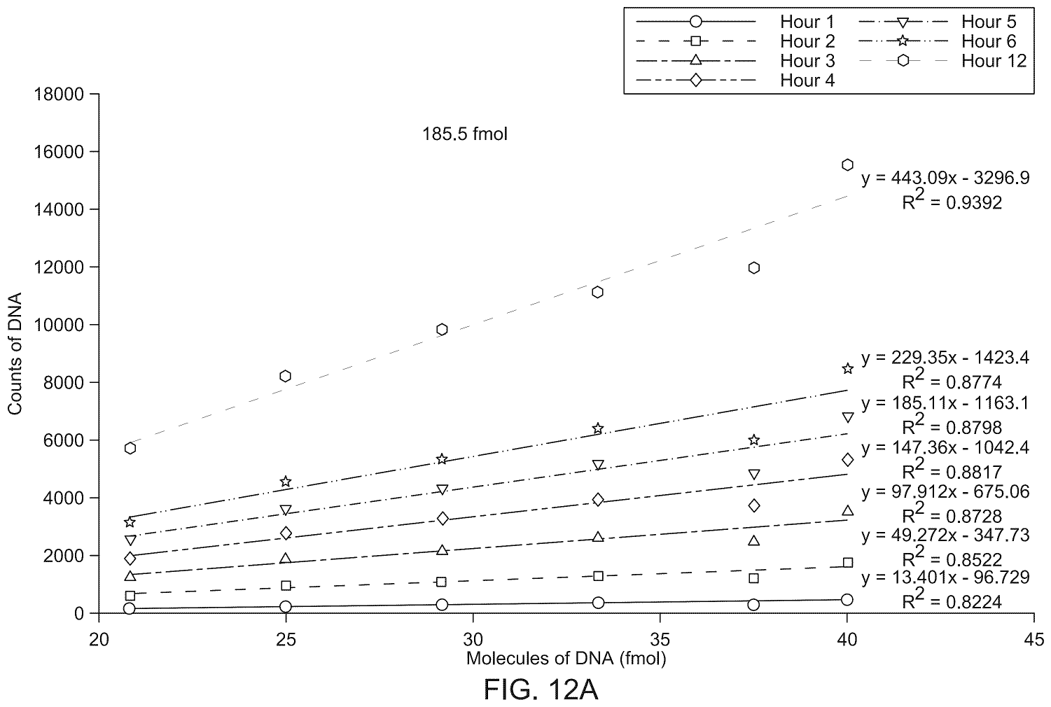

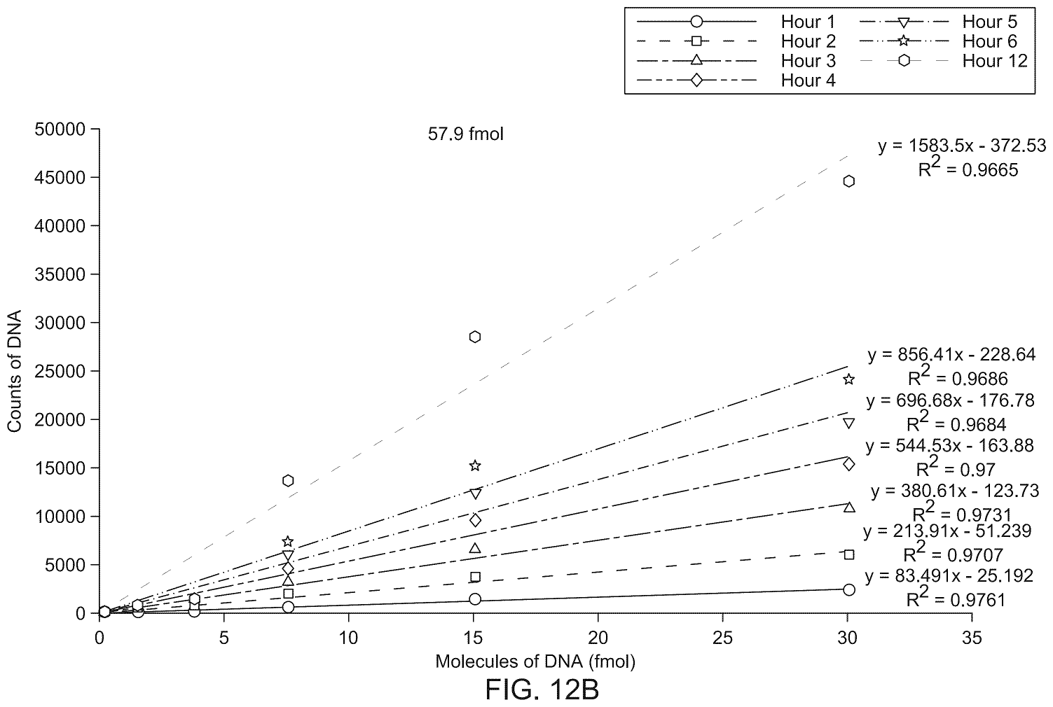

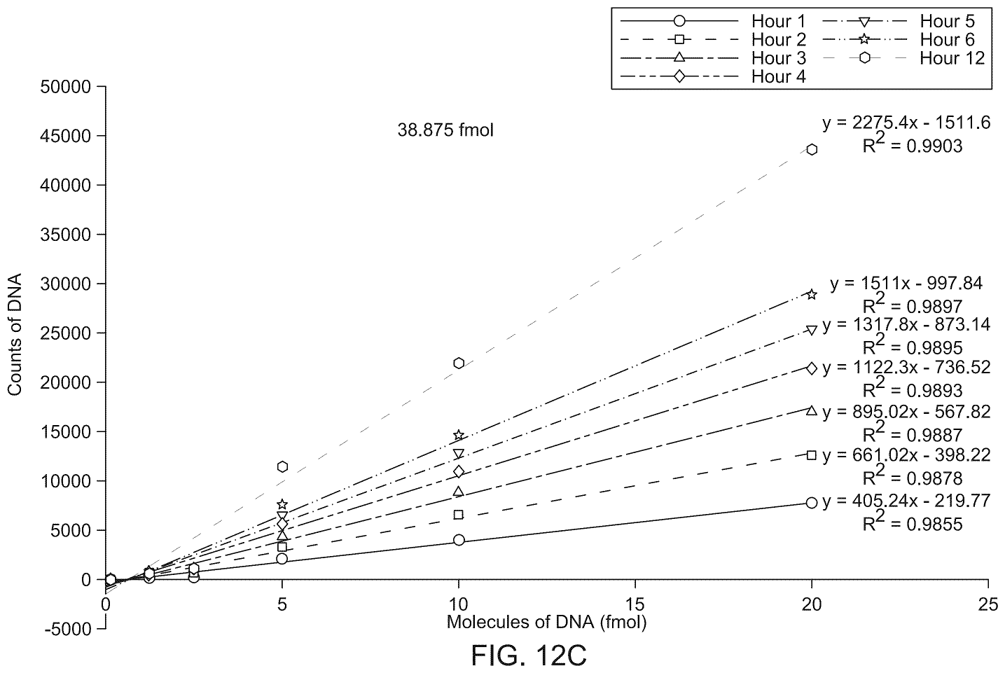

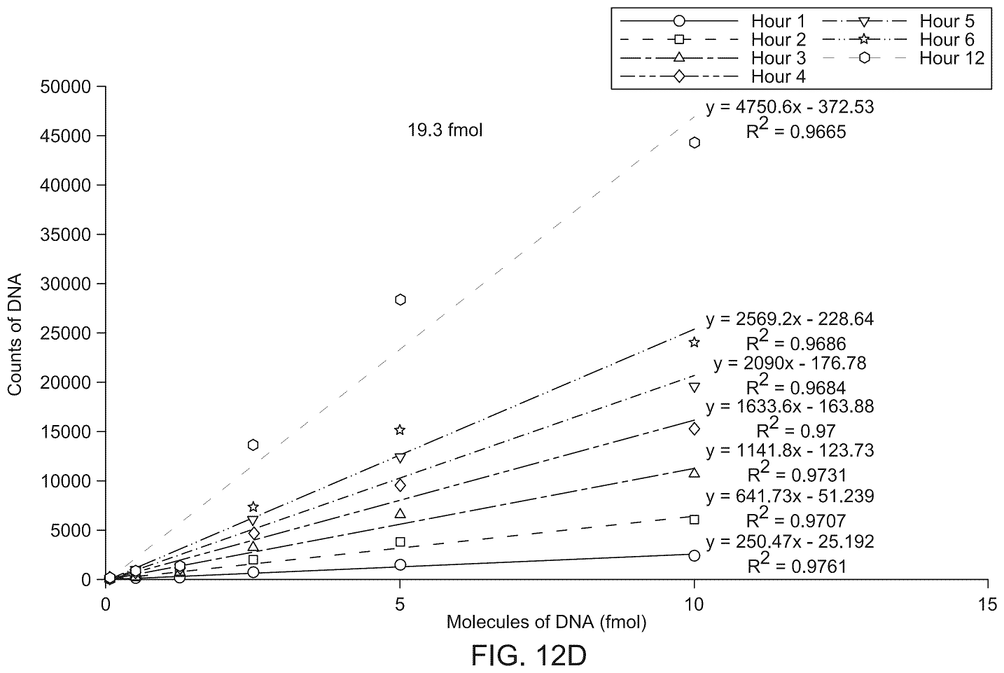

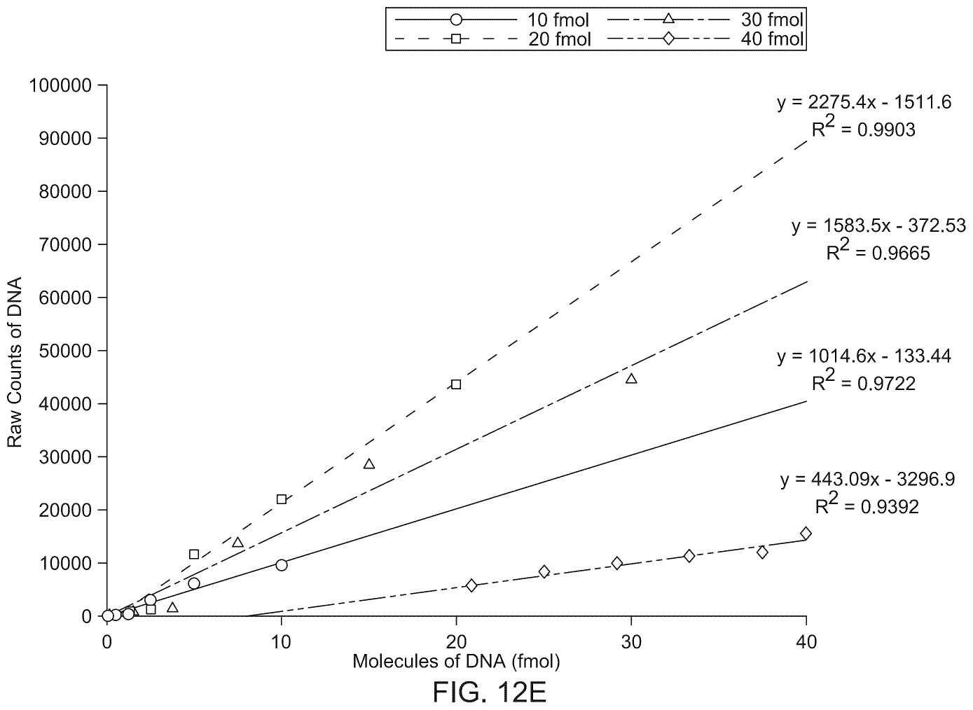

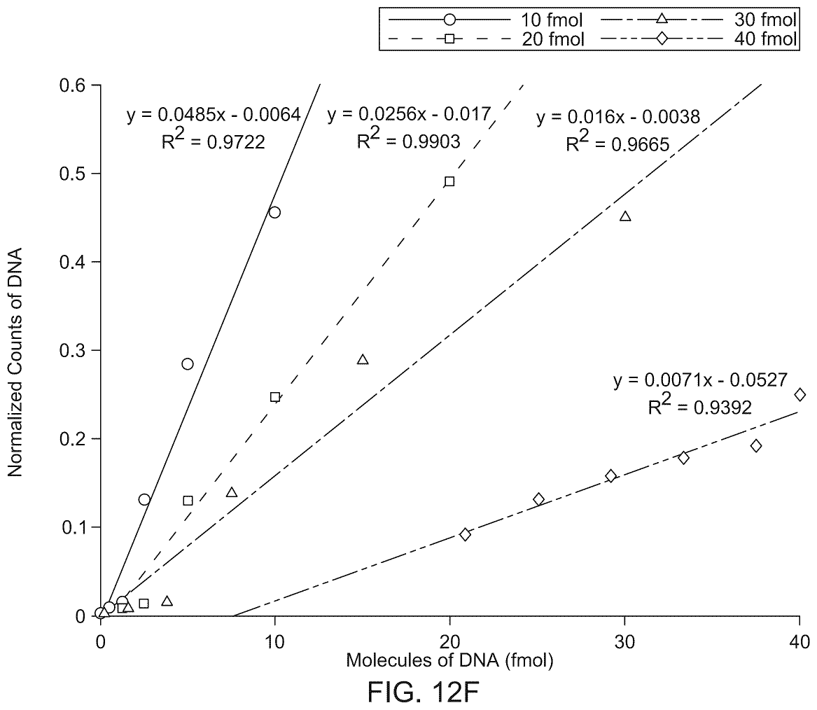

To investigate the sensitivity of using sequencing as a signal readout, different formulations of predefined mixtures of barcoded DNA without ligated substrate were tested, where each concentration corresponded to a particular barcode. It was hypothesized that nanopore sequencing would obey the coupon collector problem, whereby, the probability of a barcoded DNA being read is dependent on the total number of reads and whether it was present at high enough concentration compared to the other barcodes. To examine this in detail, calibration curves for Counts of DNA vs Molecules of DNA were generated for each of the formulations to compare hours 1-6 and 12 of sequencing, FIG. 12A-D. What was observed for all formulations was that as the sequencing run time increased, the slope of the calibration curved increased as well, with a greater than 10× increase for the 10, 30, and 40 fmol formulations and a 5× increase in the 20 fmol formulation. This showed that as the sequencing run time increased, there was an overall increase in sensitivity since the slope of the calibration lines increased as well. We further generated a scatter plot of counts normalized vs amount of DNA loaded at hour 12 to see how the sensitivity of the device compared between the different flow cells, FIG. 12F. When the counts were normalized a correlation between sensitivity and the total DNA loaded was observed; specifically, as the dynamic range decreased, the sensitivity increased. This increase in sensitivity enabled the detection of quantities lower than 1 fmol and across three orders of magnitude.

### Comparing the DzNanoporeSeq Method to Fluorescence Dz-Based Detection.

TABLE 2

| Metal | | | | | Fluorescence | |
|---|---|---|---|---|---|---|
| Ion | [Mⁿ⁺] | | DzNanoporeSeq | | Spectroscopy | |
| Li⁺ | 5 | mM | 5.00 ± 0.17 | mM | 4.04 ± 0.43 | mM |
| | 50 | mM | 50.44 ± 0.12 | mM | 56.72 ± 0.73 | mM |
| Ca²⁺ | 5 | mM | 5.246 ± 0.003 | mM | 6.89 ± 0.57 | mM |
| | 50 | mM | 50.14 ± 0.40 | mM | 55.51 ± 5.82 | mM |
| Pb²⁺ | 5 | nM | 4.95 ± 0.12 | nM | 14.53 ± 1.22 | nM |
| | 70 | nM | 71.02 ± 0.51 | mM | 69.50 ± 0.98 | nM |

*For DzNanoporeSeq n = 2. For Fluorescence spectroscopy n = 3.

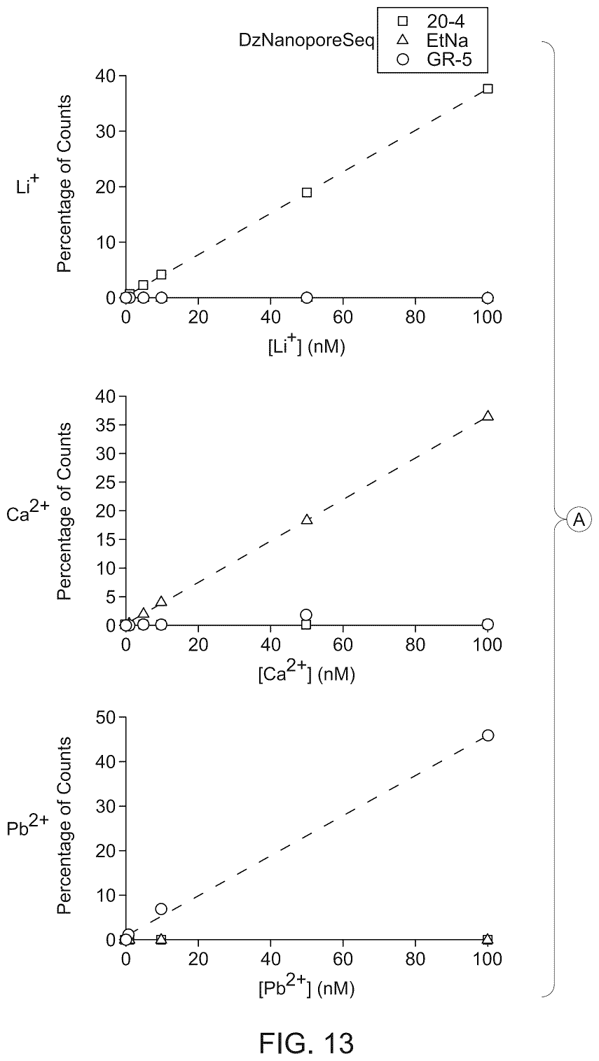

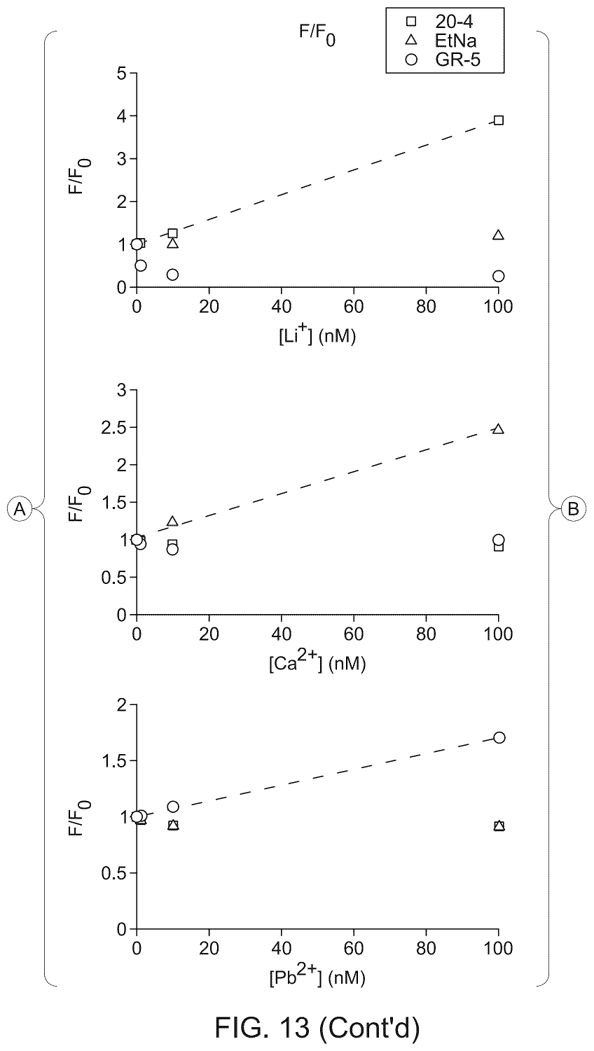

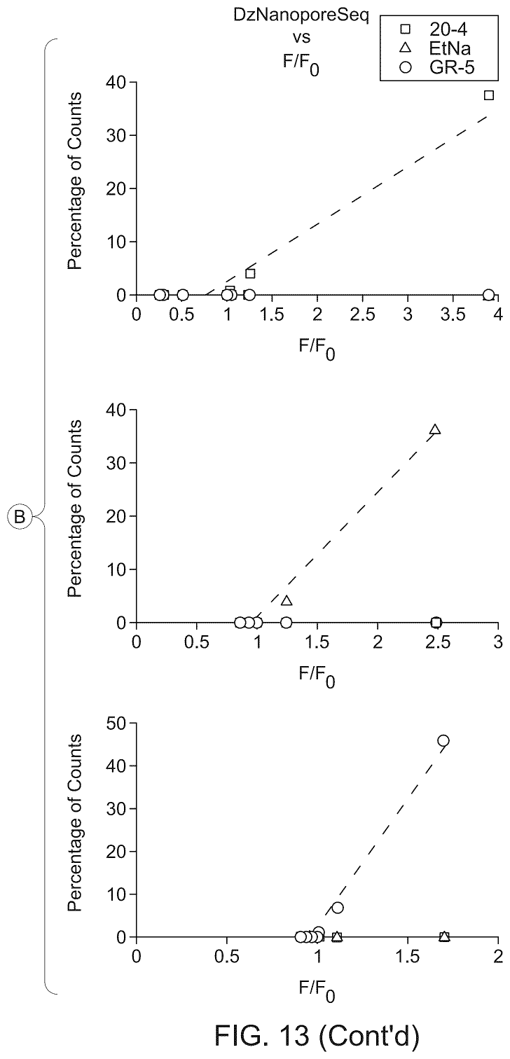

To benchmark the DzNanoporeSeq method, the same concentrations of metal ions were tested with the DzNanoporeSeq method and compared to those tested by the fluorescence method using a fluorescent reporter substrate, FIG. 13. It was observed that the DzNanoporeSeq method showed very little cross-reactivity between the three DNAzymes, which agreed with the fluorescence data. Interestingly, the 5 and 50 mM validation solutions for Li⁺ were determined to be 5.26 and 51.06 mM by the DzNanoporeSeq method and 4.04 and 56.72 mM by the fluorescence method, Table 3.1. The validation solutions for Ca²⁺ were determined to be 5.00 and 49.99 mM by the DzNanoporeSeq method and 6.89 and 55.51 mM by the fluorescence method, Table 3.1. The validation solutions for Pb²⁺ were determined to be 4.99 and 67.58 nM by the DzNanoporeSeq method and 14.53 and 69.50 nM by the fluorescence method, Table 3.1. This shows that the DzNanoporeSeq method can effectively determine metal concentrations from the internal calibration curve and is more accurate compared to the traditional fluorescence spectroscopy method, since the concentrations determined by DzNanoporeSeq were closer to the test concentration compared to the fluorescence spectroscopy method, e.g., the 5 nM Pb²⁺ was determined to be 4.95±0.12 nM by DzNanoporeSeq in comparison to 14.53±1.22 nM. Additionally, the precision improved as well. Finally, when comparing the percentage of counts to F/F₀, FIG. 13, it is observed that for all metals, there is a linear correlation between DzNanoporeSeq and the fluorescence method for monitoring DNAzyme activity.

### Scalability

Since sensitivity is dependent on the amount of DNA sequenced, threshold testing was conducted in each experiment, where instead of having one internal standard calibration curve, a single internal standard was included at a desirable threshold (e.g., the acceptable amount of metal in drinking water). This would also further aid in the scalability of DzNanoporeSeq. The threshold for Pb²⁺ was 70 nM as this approximates the EPA maximum concentration of lead, which is 72 nM.²² The thresholds tested for both Li⁺ and Ca²⁺ were 1 mM, as these were approximately the limit-of-detections (LODs) for the 20-4 and EtNa DNAzymes when using the fluorescence method. The results of each threshold test for all the runs can be found in Table 3.2. Interestingly, there was high precision in the percentage of counts for a particular threshold concentration between each experiment. This shows minimal variability in

TABLE 3

| | 1 mM Li⁺ | | 1 mM Ca²⁺ | | 70 nM Pb²⁺ | | |
|---|---|---|---|---|---|---|---|
| Internal | Experiment | | Experiment | | Experiment | | |
| Standard | 1 | 2 | 1 | 2 | 1 | 2 | Average |
| ≥1 mM Li+ | 1.59 | 1.45 | 3.12 | 3.28 | 2.94 | 3.56 | 2.70 ± 0.14 |
| ≥1 mM Ca²⁺ | 3.18 | 3.18 | 1.32 | 1.41 | 4.18 | 2.86 | 2.52 ± 0.23 |
| ≥70 nM Pb²⁺ | 31.29 | 33.05 | 31.9 | 33.83 | 34.74 | 36.33 | 33.38 ± 0.20 |

this method, since the percentage of counts for a threshold concentration has minimal variability between experiments. This in turn shows that DzNanoporeSeq has highly scalable potential.

All combinations of method or process steps as used herein can be performed in any order, unless otherwise specified or clearly implied to the contrary by the context in which the referenced combination is made.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all subranges between (and inclusive of) the minimum value of 1 and the maximum value of 10; that is, all subranges beginning with a minimum value of 1 or more (e.g., 1 to 6.1), and ending with a maximum value of 10 or less (e.g., 2.3 to 9.4, 3 to 8, 4 to 7), and finally to each number 1, 2, 3, 4, 5, 6, 7, 8, 9, and 10 contained within the range.

The compositions, systems, and corresponding methods of the present disclosure can comprise, consist of, or consist essentially of the essential elements and limitations of the disclosure as described herein, as well as any additional or optional ingredients, components, or limitations described herein or otherwise useful in the general inventive concepts.

The compositions of the present disclosure may also be substantially free of any optional or selected component or feature described herein, provided that the remaining composition still contains all of the required elements or features as described herein. In this context, and unless otherwise specified, the term “substantially free” means that the selected composition contains less than a functional amount of the optional component, typically less than 0.1% by weight, and also including zero percent by weight of such optional or selected component.

To the extent that the terms “include,” “includes,” or “including” are used in the specification or the claims, they are intended to be inclusive in a manner similar to the term “comprising” as that term is interpreted when employed as a transitional word in a claim. Furthermore, to the extent that the term “or” is employed (e.g., A or B), it is intended to mean “A or B or both A and B.” When the Applicant intends to indicate “only A or B but not both,” then the term “only A or B but not both” will be employed. Thus, use of the term “or” herein is the inclusive, and not the exclusive use. In the present disclosure, the words “a” or “an” are to be taken to include both the singular and the plural. Conversely, any reference to plural items shall, where appropriate, include the singular.

In some aspects, it may be possible to utilize the various inventive concepts in combination with one another. Additionally, any particular element recited as relating to a particularly disclosed embodiment should be interpreted as available for use with all disclosed embodiments, unless incorporation of the particular element would be contradictory to the express terms of the embodiment. Additional advantages and modifications will be readily apparent to those skilled in the art. Therefore, the disclosure, in its broader aspects, is not limited to the specific details presented therein, the representative apparatus, or the illustrative examples shown and described. Accordingly, departures may be made from such details without departing from the spirit or scope of the general inventive concepts.

Reference is made to one or more articles, patents, patent applications, or other publications, the entire content of which are expressly incorporated herein as if recited in their entirety.

While the invention has been illustrated and described in detail in the drawings and foregoing description, the same is to be considered as illustrative and not restrictive in character. It should be understood that only the exemplary embodiments have been shown and described and that all changes and modifications that come within the spirit of the invention are desired to be protected.

1. A field depolyable system for real-time detection of a plurality of metal ions in a sample, the system comprising
a multiplexed DNA zyme array comprising a plurality of DNA zyme sensors sensitive to at least one predetermined metal ion,
wherein each DNAzyme sensor comprises a DNAzyme region, a barcode region, an optional detection enhancement sequence (the 200 bp segment added for detection), and a removal tag,
means for removing unreacted DNA zyme substrate, and
means for sequencing a DNA zyme-metal sample.

2. The field depolyable system of claim 1, wherein the at least one predetermined metal ion is selected from the group comprising Li⁺, Na⁺, Ag⁺, UO₂²⁺, Cu²⁺, Cd²⁺, Ca²⁺, Hg²⁺, Mn²⁺, Pb²⁺, Tl³⁺, Ln³⁺, Cr³⁺, Cr⁶⁺, Ni²⁺, As³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, and Lu³⁺.

3. The field depolyable system of claim 1, wherein the at least one predetermined metal ion is selected from Li⁺, Na⁺, K⁺, Ag⁺, Mg²⁺, Ca²⁺, Zn²⁺, and Pb²⁺ ions.

4. The field depolyable system of claim 1, wherein the DNA zyme array comprises at least one DNA zyme selected from 17E, GR-5, EtNa, Ag10c, and NaA 43.

5. The field deployable system of claim 1, wherein the removal tag comprises biotin.

6. The field depolyable system of claim 1, wherein the means for removing unreacted DNA zyme substrate comprises streptavidin coated metal beads.

7. The field depolyable system of claim 1, wherein the barcoded region comprises a nucleotide sequence.

8. The field depolyable system of claim 1, comprising a barcode sequence unique to each of the at least one predetermined metal ion.

9. The field depolyable system of claim 1, wherein the means for sequencing a DNA zyme metal sample comprises a nanopore sequencing device.

10. A method for the detection of a plurality of metal ions in a sample, the method comprising obtaining a sample comprising at least one metal ion,
contacting the sample with a DNA zyme array to form a metal assay mixture,
optionally adding a means for removing unreacted DNA zyme substrate,
sequencing the metal assay mixture to generate sample sequence data,
comparing the sample sequence data to one or more predetermined metal ion profiles, and
determining the presence of one or more metal ions in the sample based on the predetermined metal profiles.

11. The method of claim 10, further comprising pooling one or more metal assay mixtures prior to sequencing the metal assay mixture.

12. The method of claim 10, wherein the at least one predetermined metal ion is selected from the group comprising Li⁺, Na⁺, Ag⁺, UO₂²⁺, Cu²⁺, Cd²⁺, Ca²⁺, Hg²⁺, Mn²⁺, Pb²⁺, Tl³⁺, Ln³⁺, Cr³⁺, Cr⁶⁺, Ni²⁺, As³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, and Lu³⁺.

13. The method of claim 12, wherein the at least one predetermined metal ion is selected from Li⁺, Na⁺, K⁺, Ag⁺, Mg²⁺, Ca²⁺, Zn²⁺, and Pb²⁺ ions.

14. The method of claim 10, wherein the DNA zyme array comprises at least one DNA zyme selected from 17E, GR-5, EtNa, Ag10c, and NaA43.

15. The method of claim 10, wherein each DNA zyme sequence comprises a DNA zyme region, a barcoded region, an optional detection enhancement sequence, and a removal tag.

16. The method of claim 10, wherein the means for removing unreacted DNA zyme substrate comprises streptavidin coated metal beads.

17. The method of claim 10, wherein the barcoded region comprises a nucleotide sequence.

18. The method of claim 10, wherein the barcoded region comprises a barcode sequence unique to each of the at least one predetermined metal ion.

19. The method of claim 10 wherein the sample is diluted into a reaction buffer prior to sequencing.

20. The method of claim 10, wherein comparing the sample sequence data to one or more predetermined metal ion profiles further comprises determining the concentration of the at least one predetermined metal ion.


SEQUENCE LISTING (42 sequences)

SEQ ID NO: 1 - DNA, 14 bp, synthetic construct
  source 1..14: mol_type = other DNA
agtccctgaatcga

SEQ ID NO: 2 - DNA, 25 bp, synthetic construct
  source 1..25: mol_type = other DNA
  modified_base 25: mod_base = OTHER; note = Black hole quencher 1 is attached to C nucleotide
tcgccatcttatagtgactcgtgac

SEQ ID NO: 3 - DNA, 20 bp, synthetic construct
  source 1..20: mol_type = other DNA
gtccatctcttatagtgaga

SEQ ID NO: 4 - DNA, 28 bp, synthetic construct
  source 1..28: mol_type = other DNA
tttcgccatcttcatagtgactcgtgac

SEQ ID NO: 5 - DNA, 19 bp, synthetic construct
  source 1..19: mol_type = other DNA
catctcttctatagtgaga

SEQ ID NO: 6 - DNA, 26 bp, synthetic construct
  source 1..26: mol_type = other DNA
tttcgccatctttagtgactcgtgac

SEQ ID NO: 7 - DNA, 18 bp, synthetic construct
  source 1..18: mol_type = other DNA
tccatctctttagtgaga

SEQ ID NO: 8 - DNA, 22 bp, synthetic construct
  source 1..22: mol_type = other DNA
tttcgccatctttagtgactcg

SEQ ID NO: 9 - DNA, 21 bp, synthetic construct
  source 1..21: mol_type = other DNA
cgtccatctctttatagtgag

SEQ ID NO: 10 - DNA, 17 bp, synthetic construct
  source 1..17: mol_type = other DNA
  modified_base 17: mod_base = OTHER; note = Iowa Black FQ is attached to C nucleotide
ggcggtactacgtgatc

SEQ ID NO: 11 - DNA, 18 bp, synthetic construct
  source 1..18: mol_type = other DNA
cgtccatctctagtgaga

SEQ ID NO: 12 - DNA, 30 bp, synthetic construct
  source 1..30: mol_type = other DNA
  modified_base 1: mod_base = OTHER; note = 6-FAM (Fluorescein) is attached to G nucleotide
  modified_base 30: mod_base = OTHER; note = Black hole quencher 1 is attached to A nucleotide
  modified_base 16: mod_base = OTHER; note = Riboadenosine
gtcacgagtcactataggaagatggcgaaa

SEQ ID NO: 13 - DNA, 30 bp, synthetic construct; identical to SEQ ID NO: 12
  source 1..30: mol_type = other DNA
  modified_base 1: mod_base = OTHER; note = Black hole quencher 1 is attached to G nucleotide
  modified_base 30: mod_base = OTHER; note = 6-FAM (Fluorescein) is attached to A nucleotide
  modified_base 16: mod_base = OTHER; note = Riboadenosine

SEQ ID NO: 14 - DNA, 26 bp, synthetic construct
  source 1..26: mol_type = other DNA
  modified_base 1: mod_base = OTHER; note = Black hole quencher 1 is attached to C nucleotide
  modified_base 26: mod_base = OTHER; note = 6-FAM (Fluorescein) is attached to A nucleotide
  modified_base 12: mod_base = OTHER; note = Riboadenosine
cgagtcactataggaagatggcgaaa

SEQ ID NO: 15 - DNA, 24 bp, synthetic construct
  source 1..24: mol_type = other DNA
  modified_base 1: mod_base = OTHER; note = 6-FAM (Fluorescein) is attached to G nucleotide
  modified_base 24: mod_base = OTHER; note = Iowa Black FQ is attached to C nucleotide
  modified_base 12: mod_base = OTHER; note = Riboadenosine
ggatcacgtataggaagtaccgcc

SEQ ID NO: 16 - DNA, 24 bp, synthetic construct
  source 1..24: mol_type = other DNA
  modified_base 1: mod_base = OTHER; note = 6-FAM (Fluorescein) is attached to T nucleotide
  modified_base 24: mod_base = OTHER; note = Black hole quencher 1 is attached to G nucleotide
  modified_base 10: mod_base = OTHER; note = Riboadenosine
tctcactataggaagagatggacg

SEQ ID NO: 17 - DNA, 26 bp, synthetic construct
  source 1..26: mol_type = other DNA
  modified_base 26: mod_base = OTHER; note = Black hole quencher 1 is attached to C nucleotide
tttcgccatctatagtgactcgtgac

SEQ ID NO: 18 - DNA, 21 bp, synthetic construct
  source 1..21: mol_type = other DNA
gtacgagagtaactgtagtgg

SEQ ID NO: 19 - DNA, 25 bp, synthetic construct
  source 1..25: mol_type = other DNA
  modified_base 1: mod_base = OTHER; note = Alexa Fluor 488 (NHS Ester) is attached to C nucleotide
  modified_base 25: mod_base = OTHER; note = Iowa Black FQ is attached to C nucleotide
  modified_base 13: mod_base = OTHER; note = Riboguanosine
ccactacagttagggactctcgtac

SEQ ID NO: 20 - DNA, 19 bp, synthetic construct
  source 1..19: mol_type = other DNA
gtccatctctatagtgaga

SEQ ID NO: 21 - DNA, 87 bp, synthetic construct
  source 1..87: mol_type = other DNA
ccgagcgaagacttgccatgtgtatgtgggagacggtcgagtgtgggctggatcattatggcaagaccataccatgtcttcgctcgg

SEQ ID NO: 22 - DNA, 87 bp, synthetic construct
  source 1..87: mol_type = other DNA
ccgagcgaagacatggtatggtcttgccataatgatccagcccacactcgaccgtctcccacatacacatggcaagtcttcgctcgg

SEQ ID NO: 23 - DNA, 50 bp, synthetic construct
  source 1..50: mol_type = other DNA
  modified_base 1: mod_base = OTHER; note = Phosphorylation
gccatgtgtatgtgggagacggtcgagtgtgggctggatcattatggcaa

SEQ ID NO: 24 - DNA, 50 bp, synthetic construct
  source 1..50: mol_type = other DNA
  modified_base 1: mod_base = OTHER; note = Phosphorylation
ggtcttgccataatgatccagcccacactcgaccgtctcccacatacaca

SEQ ID NO: 25 - DNA, 30 bp, synthetic construct
  source 1..30: mol_type = other DNA
  modified_base 1: mod_base = OTHER; note = Phosphorylation
cccgcgaaattaatacgactcactataggg

SEQ ID NO: 26 - DNA, 35 bp, synthetic construct
  source 1..35: mol_type = other DNA
  modified_base 1: mod_base = OTHER; note = Phosphorylation
tggcccctatagtgagtcgtattaatttcgcggga

SEQ ID NO: 27 - DNA, 29 bp, synthetic construct
  source 1..29: mol_type = other DNA
  modified_base 1: mod_base = OTHER; note = Phosphorylation
gaccagatccggctgctaacaaagcccga

SEQ ID NO: 28 - DNA, 55 bp, synthetic construct
  source 1..55: mol_type = other DNA
  modified_base 1: mod_base = OTHER; note = Biotin-TEG is attached to T nucleotide
  modified_base 9: mod_base = OTHER; note = Uracil
tctcactattaggaagagatggacgttttttcgggctttgttagcagccggatct

SEQ ID NO: 29 - DNA, 109 bp, synthetic construct
  source 1..109: mol_type = other DNA
cccgcgaaattaatacgactcactataggggccatgtgtatgtgggagacggtcgagtgtgggctggatcattatggcaagaccagatccggctgctaacaaagcccga

SEQ ID NO: 30 - DNA, 139 bp, synthetic construct
  source 1..139: mol_type = other DNA
  modified_base 1: mod_base = OTHER; note = Biotin-TEG is attached to T nucleotide
  modified_base 9: mod_base = OTHER; note = Uracil
tctcactataggaagagatggacgttttttcgggctttgttagcagccggatctggtcttgccataatgatccagcccacactcgaccgtctcccacatacacatggcccctatagtgagtcgtattaatttcgcggga

SEQ ID NO: 31 - DNA, 38 bp, synthetic construct
  source 1..38: mol_type = other DNA
ccgagcgaagacaagccatgtgtatgtgggagacggtc

SEQ ID NO: 32 - DNA, 46 bp, synthetic construct
  source 1..46: mol_type = other DNA
ttgccatgtgtatgtgggagacggtcgagtgtgggctggatcatta

SEQ ID NO: 33 - DNA, 46 bp, synthetic construct
  source 1..46: mol_type = other DNA
taatgatccagcccacactcgaccgtctcccacatacacatggcaa

SEQ ID NO: 34 - DNA, 44 bp, synthetic construct
  source 1..44: mol_type = other DNA
ccgaccgaagacatggtcttgccataatgatccagcccacactc

SEQ ID NO: 35 - DNA, 38 bp, synthetic construct
  source 1..38: mol_type = other DNA
ccgagcgaagacttgccatgtgtatgtgggagacggtc

SEQ ID NO: 36 - DNA, 45 bp, synthetic construct
  source 1..45: mol_type = other DNA
ccgagcgaagacatggtcttgccatgaatgatccagcccacactc

SEQ ID NO: 37 - DNA, 24 bp, synthetic construct
  source 1..24: mol_type = other DNA
aaattaatacgactcactataggg

SEQ ID NO: 38 - DNA, 25 bp, synthetic construct
  source 1..25: mol_type = other DNA
tggcccctatagtgagtcgtattaa

SEQ ID NO: 39 - DNA, 54 bp, synthetic construct
  source 1..54: mol_type = other DNA
  modified_base 1: mod_base = OTHER; note = Biotin-TEG is attached to T nucleotide
  modified_base 10: mod_base = OTHER; note = Riboadenosine
tctcactataggaagagatggacgttttttcgggctttgttagcagccggatct

SEQ ID NO: 40 - DNA, 34 bp, synthetic construct
  source 1..34: mol_type = other DNA
gtccatctctcgatcagcaaccgagatagtgaga

SEQ ID NO: 41 - DNA, 45 bp, synthetic construct
  source 1..45: mol_type = other DNA
tccatctcttttctcacagcgtactcgctaaggttgttagtgaga

SEQ ID NO: 42 - DNA, 35 bp, synthetic construct
  source 1..35: mol_type = other DNA
gtccatctctgaagtagcgccgccgtatagtgaga